(19) United States
(12) Patent Application Publication
(10) Pub. No.: US 2023/0017157 A1
(43) Pub. Date: Jan. 19, 2023

(54) METHOD AND DEVICE FOR PROTECTION OF MEDICAL DEVICES FROM ANOMALOUS INSTRUCTIONS

(71) Applicant: FLOWHOW LTD., Jerusalem (IL)

(72) Inventors: Tom MAHLER, Tel Aviv (IL); Yuval SHAHAR, Omer (IL); Yuval ELOVICI, Arugot (IL)

(21) Appl. No.: 17/779,888

(22) PCT Filed: Nov. 26, 2020
(86) PCT No.: PCT/IL2020/051222

§ 371 Date: May 25, 2022

Related U.S. Application Data
(60) Provisional application No. 63/018,093, filed on Apr. 30, 2020.
(60) Provisional application No. 62/940,927, filed on Nov. 27, 2019.

(51) Int. Cl. G16H 50/20; G06T 7/00

(52) U.S. Cl. CPC G16H 50/20; G06T 7/0012; G06T 2207/20081

(57) ABSTRACT

Provided herein are a method and device for detection of anomalous instructions sent from a controller of a medical device, to be received by a medical device. The method and the device utilize a dual layer architecture including a first, unsupervised detection layer and a second, supervised detection layer, wherein the layers are applied to the received instructions in series to efficiently detect anomalous instruction prior to the instructions reaching the medical device.

### TECHNICAL FIELD

The present disclosure relates generally to devices and methods for detection of anomalous instructions sent from a controller of a medical device to the medical device, utilizing machine learning models.

### BACKGROUND

Complex medical devices (e.g., medical imaging devices (MIDs)) often consist of an entire ecosystem of connected components (e.g., data processing servers, physical components, etc.), and are commonly controlled by instructions (or in other words, instruction sets) sent from a controller, such as, for example, a host PC. The anomalous instructions can introduce one or more potentially harmful threats to patients (e.g., radiation overexposure), one or more potentially harmful threats to physical components (e.g., manipulation of device motors) devices, and/or one or more potentially harmful threats to functionality (e.g., manipulation of medical images).

For example, some of the high-risk vulnerabilities to medical devices include allowing an attacker to execute privileged instructions and to use hard-coded credentials that could impact the system integrity and availability. In another example, an adversarial attacker could tamper with medical images to insert or remove tumors. Additionally, human errors (e.g., a technician's configuration mistake) and software bugs may also result in anomalous instructions. For example, incorrect settings on the CT controller (pr host PC) may result in radiation overexposure of a plurality of patients for extended periods of time, such as months. In another example, a critical software bug in a radiation therapy device for the treatment of cancer can result in patients receiving massive amounts of direct radiation (sometimes a hundred times more than the usual dose) that may even lead to death.

Existing methods for mitigating the risk of anomalous instructions from cyber-attacks commonly are aimed on protecting the host PC from the hospital networks. Such methods are limited and are often breached, as they rely on constantly installing regular security updates, a challenging task in a clinical setting with numerous out-of-date devices.

Anomaly detection is commonly used for various applications, such as fraud, intrusion detection, sensor networks and Internet of Things (IoT). Anomaly detection can use supervised methods or unsupervised methods. Using supervised anomaly detection methods requires data labeling (often by domain experts), an expensive and time-consuming task. Unsupervised anomaly detection methods can be used instead, as they find patterns that do not conform to an expected behavior; thus, they can also find new, unknown, anomalies.

There is a thus need in the art for devices and methods allowing the efficient and reliable detection of anomalous instructions sent from a controller of a medical device to the medical device and to further prevent the anomalous instructions from reaching the medical device.

### SUMMARY

Aspects of the disclosure, according to some embodiments thereof, relate to advantageous devices and methods for identification or detection of anomalous instructions sent form a controller of a medical device to the medical device, utilizing a dual layer architecture including a plurality of algorithms, configured to identify context free and context sensitive anomalous instructions, wherein the detection layer operate in series, to provide an effective, reliable and efficient detection of such anomalous instructions. In some embodiments, the advantageous devices and methods may further prevent the identified anomalous instructions from reaching the medical device, to thereby prevent potential harmful consequences associated therewith.

According to some embodiments, provided herein are advantageous methods, devices, and non-transitory computer-readable medium, which utilize dual layer detection architecture for the protection of medical devices from anomalous instructions sent from a controller (such as, a host PC).

According to some embodiments, there is provided a method for detection of anomalous instructions sent from a controller to a corresponding medical device, the method includes the steps of: receiving instructions sent from the controller, the instruction being intended to be received by the medical device; and analyzing the instructions by applying: a first detection layer, the first detection layer is or includes an unsupervised machine learning model configured to detect context free (CF) anomalous instructions; and a second detection layer, which includes a supervised machine learning model configured to detect context sensitive (CS) anomalous instructions; wherein the second layer is applied to instructions that were not detected as anomalous by the first detection layer.

According to some embodiments, there is provided a device for detection of anomalous instructions sent form a controller to a medical device, the device includes a processor configured to at least receive instructions from the controller, the instructions being intended to be received by the medical device; and analyze the instructions by applying: a first detection layer which is or includes an unsupervised detection layer machine learning model configured to detect context free (CF) anomalous instructions; and a second detection layer which is or includes a supervised detection layer machine learning model configured to detect context sensitive (CS) anomalous instructions, wherein the first and second detection layers are applied consecutively, whereby the second layer is applied to instructions that were not detected as anomalous by the first detection layer.

According to some embodiments, there is provided a non-transitory computer-readable medium having stored thereon instructions that cause a processor to receive instructions sent from a controller, the instructions being intended to be received by a medical device; and analyze the instructions by applying: a first detection layer to the received instructions, the first detection layer being an unsupervised detection layer include machine learning model configured to detect context free (CF) anomalous instructions; and a second detection layer, the second detection layer being a supervised detection layer includes machine learning model, configured to detect context sensitive (CS) anomalous instructions, wherein the second layer is applied to instructions that were not detected as anomalous by the first detection layer.

According to some embodiments, there is provided a method for detection of anomalous instructions sent from a controller to be received by a medical device, the method including receiving instructions sent from the controller, the instruction being intended to be received by the medical device, and analyzing the instructions by applying:

(1) a first detection layer, the first detection layer including an unsupervised machine learning model configured to detect context free (CF) anomalous instructions, and (2) a second detection layer, the second detection layer including a supervised machine learning model configured to detect context sensitive (CS) anomalous instructions, wherein the second layer is applied to instructions that were not detected as anomalous by the first detection layer.

According to some embodiments, there is provided a device for detection of anomalous instructions sent form a controller to a medical device, the device including:

a processor configured to: receive instructions from the controller, the instructions being intended to be received by the medical device, and analyze the instructions by applying:

(1) a first detection layer including an unsupervised detection layer machine learning model configured to detect context free (CF) anomalous instructions, and (2) a second detection layer including a supervised detection layer machine learning model configured to detect context sensitive (CS) anomalous instructions, wherein the first and second detection layers are applied consecutively, whereby the second layer is applied to instructions that were not detected as anomalous by the first detection layer.

According to some embodiments, there is provided a non-transitory computer-readable medium having stored thereon instructions that cause a processor to: receive instructions sent from a controller, the instructions being intended to be received by a medical device, and analyze the instructions by applying: (1) a first detection layer to the received instructions, the first detection layer being an unsupervised detection layer includes machine learning model configured to detect context free (CF) anomalous instructions, and (2) a second detection layer, the second detection layer being a supervised detection layer includes machine learning model, configured to detect context sensitive (CS) anomalous instructions, wherein the second layer is applied to instructions that were not detected as anomalous by the first detection layer.

According to some embodiments, the first detection layer and the second detection layer are applied in series.

According to some embodiments, the detection of the anomalous instructions is in real time.

According to some embodiments, analyzing by applying the first detection layer includes calculating an anomaly score of the received instructions and comparing the anomaly score with an anomaly threshold.

According to some embodiments, the anomaly score is associated with an expected proportion of anomalies in the instructions, generated during training of the detection layer.

According to some embodiments, applying the first detection layer includes applying one or more of algorithms to the received instructions, wherein each algorithm outputs a score associated with at least one of the received instructions and a potential level of anomaly of the received instructions.

According to some embodiments, calculating an anomaly score includes selecting the anomaly score from the scores outputted by the plurality of algorithms associated with the first detection layer.

According to some embodiments, the comparison between the anomaly score and the anomaly threshold is associated with one or more of: a deviation from a predetermined threshold value, a deviation from a corresponding standard parameter value, an unlikely parameter value, and an unlikely combination of parameter value.

According to some embodiments, applying the first layer includes determining if one or more parameter values of the received instructions deviate from values of corresponding parameters of a predetermined parameter value data set, wherein a deviation between the one or more parameter values of the received instructions and values of parameters in the predetermined value data set is indicative of the instructions being anomalous.

According to some embodiments, the context sensitive (CS) anomalous instructions relate to one or more context values associated with the received instructions and to a specific patient intended to be monitored or treated by the medical device by implementing the received instructions.

According to some embodiments, the one or more context values related to the specific patient are selected from: type of medical procedure applied to the patient, a selected scan option, region of interest of the patient body being monitored or treated, a study, and a protocol of the instructions.

According to some embodiments, the one or more context values related to the specific patient includes characteristics of the specific patient, selected from: age, gender, weight, and medical history.

According to some embodiments, the method further includes receiving a context value associated with the received instructions, and wherein analyzing by applying the second detection layer includes: (i) applying the received instructions to at least one supervised classification algorithm configured to output a predicted context value associated with the received instructions, and (ii) comparing the predicted context value with the received context value.

According to some embodiments, the second layer is configured to detect the instructions as anomalous if the predicted context value is essentially unequal to the received context value.

According to some embodiments, the medical device is a medical imaging device (MID).

According to some embodiments, the MID is selected from CT, MM, X-Ray generator (digital radiography), Ultrasound, SPECT, and PET.

According to some embodiments, the medical device is a CT and the context value is selected from: amount of radiation, and exposure time.

According to some embodiments, the controller includes a host PC.

According to some embodiments, the method further includes issuing an alert if anomalous instruction(s) have been identified.

According to some embodiments, the method further includes preventing or blocking a detected anomalous instruction from reaching the medical device.

According to some embodiments, the method further includes identifying the detected anomalous instructions as at least one specified type of anomalous instructions.

According to some embodiments, identifying the detected anomalous instructions as at least one specified type of anomalous instructions is based, at least in part, on an irregularity identified by at least one of the first detection layer and the second detection layer.

According to some embodiments, identifying the detected anomalous instructions as at least one specified type of anomalous instructions includes, at least in part, classifying an irregularity identified by at least one of the first detection layer and the second detection layer into at least one irregularity type classification.

According to some embodiments, the method includes outputting recommended instructions based, at least in part, on at least one of the detected anomalous instructions and the type of anomalous instructions.

According to some embodiments, the method includes outputting a signal to the medical device including instructions based, at least in part, on at least one of the detected anomalous instructions and the type of anomalous instructions, wherein the signal is associated with remediation instructions configured to replace the anomalous instructions.

According to some embodiments, the method includes generating remediation instructions configured to replace the anomalous instructions.

According to some embodiments, the method includes detecting an error associated with the generation of the received instructions from the controller using one or more values associated with the detected anomalous instructions, and wherein the generation is automatic and/or manual.

According to some embodiments, the anomalous instructions result from cyber-attack, operator error and/or internal software bugs.

According to some embodiments, the method further includes assigning a risk score to the detected anomalous instructions associated with a severity level of the detected anomalous instructions.

According to some embodiments, the instructions are received using a hypervisor algorithm configured to apply the received instructions to a virtual machine (VM).

According to some embodiments, the first detection layer and the second detection layer are applied in series.

According to some embodiments, the detection of the anomalous instructions is in real time.

According to some embodiments, analyzing by applying the first detection layer includes calculating an anomaly score of the received instructions and comparing the anomaly score with an anomaly threshold.

According to some embodiments, the anomaly score is associated with an expected proportion of anomalies in the instructions, generated during training of the detection layer.

According to some embodiments, applying the first detection layer includes applying a one or more of algorithms to the received instructions, wherein each algorithm outputs a score associated with at least one of the received instructions and a potential level of anomaly of the received instructions.

According to some embodiments, calculating an anomaly score includes selecting the anomaly score from the scores outputted by the plurality of algorithms associated with the first detection layer.

According to some embodiments, the comparison between the anomaly score and the anomaly threshold is associated with one or more of: a deviation from a predetermined threshold value, a deviation from a corresponding standard parameter value, an unlikely parameter value, and an unlikely combination of parameter value.

According to some embodiments, applying the first layer includes determining if one or more parameter values of the received instructions deviate from values of corresponding parameters of a predetermined parameter value data set, wherein a deviation between the one or more parameter values of the received instructions and values of parameters in the predetermined value data set is indicative of the instructions being anomalous.

According to some embodiments, the context sensitive (CS) anomalous instructions relate to one or more context values associated with the received instructions and to a specific patient being potentially (/intended to be) monitored or treated by the medical device by implementing the received instructions.

According to some embodiments, the one or more context values related to the specific patient are selected from: type of medical procedure applied to the patient, a selected scan option, region of interest of the patient body being monitored or treated, a study, and a protocol of the instructions.

According to some embodiments, the one or more context values related to the specific patient includes characteristics of the specific patient, selected from: age, gender, weight, and medical history.

According to some embodiments, the processor is further configured to receive a context value associated with the received instructions, and wherein analyzing by applying the second detection layer includes: applying the received instructions to at least one supervised classification algorithm configured to output a predicted context value associated with the received instructions, and comparing the predicted context value with the received context value.

According to some embodiments, the second layer is configured to detect the instructions as anomalous if the predicted context value is essentially unequal to the received context value.

According to some embodiments, the medical device is a medical imaging device (MID).

According to some embodiments, the MID is selected from CT, MM, X-Ray generator (digital radiography), Ultrasound, SPECT, and PET.

According to some embodiments, the medical device is a CT and the context value is selected from: amount of radiation, and exposure time.

According to some embodiments, the controller includes a host PC.

According to some embodiments, the processor is further configured to issue an alert if anomalous instruction(s) have been identified.

According to some embodiments, the processor is further configured to prevent or block a detected anomalous instruction from reaching the medical device.

According to some embodiments, the processor is further configured to identify the detected anomalous instructions as at least one specified type of anomalous instructions.

According to some embodiments, identifying the detected anomalous instructions as at least one specified type of anomalous instructions is based, at least in part, on an irregularity identified by at least one of the first detection layer and the second detection layer.

According to some embodiments, identifying the detected anomalous instructions as at least one specified type of anomalous instructions includes, at least in part, classifying an irregularity identified by at least one of the first detection layer and the second detection layer into at least one irregularity type classification.

According to some embodiments, the processor is further configured to output recommended instructions based, at least in part, on at least one of the detected anomalous instructions and the type of anomalous instructions.

According to some embodiments, the processor is further configured to output a signal to the medical device including instructions based, at least in part, on at least one of the detected anomalous instructions and the type of anomalous instructions, wherein the signal is associated with remediation instructions configured to replace the anomalous instructions.

According to some embodiments, the processor is further configured to generate remediation instructions configured to replace the anomalous instructions.

According to some embodiments, the processor is further configured to detect an error associated with the generation of the received instructions from the controller using one or more values associated with the detected anomalous instructions, and wherein the generation is automatic and/or manual.

According to some embodiments, the anomalous instructions result from cyber-attack, operator error and/or internal software bugs.

According to some embodiments, the processor is further configured to assign a risk score to the detected anomalous instructions associated with a severity level of the detected anomalous instructions.

According to some embodiments, the device includes one or more of: a communication unit, a power source, a display, a user interface, an alert unit. According to some embodiments, the device is further configured to issue an alert if anomalous instructions have been identified.

According to some embodiments, the device is further configured to couple to the controller at a first end thereof and couple to the medical device at a second end thereof.

According to some embodiments, the device is further configured to wirelessly communicate with at least one of the controller and the medical device.

According to some embodiments, the instructions are received using a hypervisor algorithm configured to apply the received instructions to a virtual machine (VM).

According to some embodiments, the device includes a unidirectional channel coupled to the processor and configured to direct the instructions in only one direction, thereby preventing one or more signals from traveling from the processor to an external device.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures: FIG. 1, which shows a flowchart of functional steps in a process for detection of anomalous instructions, in accordance with some embodiments of the present invention;

FIG. 2, which shows a schematic illustration of an exemplary device for detection of anomalous instructions, in accordance with some embodiments of the present invention;

FIG. 3 shows a schematic illustration of an exemplary system for detection and prevention of malicious instructions, in accordance with some embodiments of the present invention;

FIG. 4 is a schematic illustration of a dual-layer architecture for the protection of medical devices from anomalous instructions, using both the context-free (CF) and the context-sensitive (CS) layers, in accordance with some embodiments of the present invention.

FIG. 5 is a table of exemplary results of the unsupervised anomaly detection for the first detection layer, in accordance with some embodiments of the present invention;

FIG. 6A, FIG. 6B, FIG. 6C, and FIG. 6D show tables of exemplary results of the dual-layer architecture, showing the performances of the first detection layer alone and with the additional second detection layers, in accordance with some embodiments of the present invention;

FIG. 7 shows exemplary results of the dual-layer architecture, showing the performance of the first detection layer alone, and with the additional second detection layers, in accordance with some embodiments of the present invention;

FIG. 8 shows a schematic data-flow diagram illustration of a medical device with a controller, according to some embodiments of the present invention;

FIG. 9 shows an exemplary schematic illustration of an out-of-band channel, in accordance with some embodiments of the present invention;

FIG. 10 shows a schematic illustration of an exemplary plugin module, in accordance with some embodiments of the present invention.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there are provided devices and methods allowing the detection of anomalous instructions sent from a controller of a medical device to the medical device, wherein the detection devices and methods utilize hybrid anomaly detection, which includes two detection layers: a first layer being an unsupervised layer and a second layer being a supervised layer, operating in serial.

Advantageously, by utilizing the two detection layers in serial, anomalous commands of various types may be detected/identified and may further be prevented from reaching the medical device.

In some embodiments, hybrid anomaly detection combines unsupervised and supervised methods, for example, e.g., using unsupervised learning to find meaningful features, and then insert them as the input of a supervised classification algorithm, and can be used to detect context sensitive anomalies. In some embodiments, hybrid anomaly detection can be used by first creating clusters using unsupervised learning and then creating an unsupervised model of the reconstruction error to detect the fraud. However, the lack of labels may make context sensitive anomaly detection harder for unsupervised methods. According to some embodiments, provided herein are method, device, and non-transitory computer-readable medium utilizing dual layer architecture for the protection of medical devices from anomalous instructions sent from a controller (such as, a host PC). According to some embodiments, the method, device, and non-transitory computer-readable medium are configured to analyze the instructions sent from the controller of the medical device to the physical components of the medical device using a dual layer architecture for the detection of anomalous instructions. According to some embodiments, the dual layer architecture is configured to analyze the instructions sent from the controller. According to some embodiments, the instructions include one or more signals intended to be received by the medical device and/or the physical components of the medical device.

According to some embodiments, the architecture includes two detection layers: (1) a first detection layer being an unsupervised detection layer configured to detect context-free (CF) anomalous instructions; and (2) a second detection later being a supervised detection layer configured to detect context-sensitive (CS) anomalous instructions. According to some embodiments, the second detection layer is applied to instructions that were not detected/identified as anomalous by the first detection layer. Thus, the second detection layer does not evaluate any instructions that were detected as anomalous by the first detection layer.

According to some embodiments, the second detection layer considerably enhances the sensitivity of the architecture in the detection of anomalous instructions, even when the original instruction was not detected as anomalous by the first detection layer. For example, anomalous instructions might not be detected as anomalous by the first detection layer but afterwards will be detected as anomalous by the second detection layer if the instruction may make sense in some potentially plausible context.

According to some embodiments, the device disclosed herein is configured such that no hardware and/or software changes need to be made to the medical device to which the device is coupled to. According to some embodiments, the device and/or the dual layer architecture is coupled to at least one isolated channel between the controller and the medical device. According to some embodiments, the device and/or the dual layer architecture is configured to receive all output commands from the controller. According to some embodiments, the device is configured to couple to a medical device such that all data being received by the medical device is first sent to the device and/or the dual layer architecture.

According to some embodiments, the dual layer architecture and/or the device are configured to couple to the medical device via a secure/private channel, thereby allowing the monitoring of the incoming commands/instructions without directly interfering with the medical device operation, thereby easing integration and regulation validations.

According to some embodiments, the dual layer architecture and/or the device are configured to couple to the medical device via one or more unidirectional links (one-way links) that physically allow data to flow only one way, such as, for example, using one or more diodes.

According to some embodiments, the device may include one or more plugin modules configured to adapt the device to one or more specified medical devices. According to some embodiments, the plugin module allows the device and/or the dual layer architecture system to fit a wide range of devices. According to some embodiments, the plugin module allows the device and/or the dual layer module to use different data and/or different algorithms. According to some embodiments, the plugin module may include data associated with one or more training set of the algorithms associated with the dual layer architecture. According to some embodiments, the plugin module may include a code/key associated with a specified medical device, wherein the code/key is used to determine which machine learning algorithms and/or which data to use for each layer of the dual layer architecture. According to some embodiments, the data may be associated with specified thresholds relating to a specific medical device. According to some embodiments, the code/key may be associated with a modality, brand, and or year of manufacturing of the medical device to which the plugin module is coupled.

According to some embodiments, the device and/or the dual architecture layer may include a data collection tool and/or backend cloud training. According to some embodiments, the data collection tool may be online of offline. According to some embodiments, the data collection tool may be configured to collect data required to train one or more of the machine learning models. According to some embodiments, the data collection tool may be configured to collect log files and traffic inside the medical device (for example, data associated with the ecosystem of the medical device).

According to some embodiments, the backend cloud training may include big data infrastructure configured to manage the collected data. According to some embodiments, the backend cloud training is configured to allow automatic pipeline training to produce improved models and test their performance.

According to some embodiments, the device and/or the dual architecture layer may include one or more anomaly explanation module. According to some embodiments, the anomaly explanation module may include one or more of a function and an algorithm configured to select/detect/identify one or more features and/or parameters associated with the detected anomalous instructions (e.g., identified wrong parameters in a protocol). According to some embodiments, the anomaly explanation module is configured to generate an output, wherein the output may provide an operator/user an explanation of why the detected anomalous command is an anomaly (e.g., direct the operator to the mistake).

According to some embodiments, the output may include a revision suggestion. According to some embodiments, the device and/or the dual architecture layer may include one or more revision suggestion modules configured to generate one or more revision suggestions associated with the detected anomalous instruction and/or the explanation of the anomaly explanation module. According to some embodiments, the revision suggestion may include one or more signals configured to fix the instructions (e.g., suggest a better-fit protocol). According to some embodiments, the revision suggestions may be based, at least in part, on predictions of trained classifiers configured to propose suggestions how to fix the anomalous commands.

According to some embodiments, the device and/or the dual architecture layer may include one or more algorithms configured to develop one or more message, such as, for example, text to be read by an operator, in which the detected anomaly is explained (for example, in a human-understandable manner). According to some embodiments, the device and/or the dual architecture layer may include an explainable module configured to generate one or more signals associated with an explanation including one or more reasons for the instructions to have been detected as anomalous.

According to some embodiments, the method may include identifying the detected anomalous instructions as at least one specified type of anomalous instructions.

According to some embodiments, the method may include identifying the detected anomalous instructions as at least one specified type of anomalous instructions is based, at least in part, on an irregularity identified by at least one of the first detection layer and the second detection layer. According to some embodiments, the method may include identifying the detected anomalous instructions as at least one specified type of anomalous instructions includes, at least in part, classifying an irregularity identified by at least one of the first detection layer and the second detection layer into at least one irregularity type classification.

According to some embodiments, the method may include outputting recommended instructions based, at least in part, on at least one of the detected anomalous instructions and the type of anomalous instructions. According to some embodiments, the method may include outputting a signal to the medical device including instructions based, at least in part, on at least one of the detected anomalous instructions and the type of anomalous instructions, wherein the signal is associated with revision suggestions configured to replace the anomalous instructions.

Reference is made to FIG. 1, which shows a flowchart of functional steps in a process for detection of anomalous instructions, in accordance with some embodiments of the present invention. According to some embodiments, anomalous instructions may result from any one or more of a cyber-attack, operator error and/or internal software bugs.

According to some embodiments, the method is configured for detection of anomalous instructions sent from a controller to be received by a medical device. According to some embodiments, the medical device may be a medical imaging device (MID). According to some embodiments, the MID is selected from CT, MRI, X-Ray generator (digital radiography), Ultrasound, SPECT, and PET. According to some embodiments, the medical device can be a CT and the context value is selected from: amount of radiation, exposure time, (others). According to some embodiments, the controller may include a host PC of the medical device.

According to some embodiments, at step 102, the method includes receiving and/or detecting instructions sent from the controller of the medical device. According to some embodiments, the received instructions are intended to be received by the medical device. According to some embodiments, the received instructions are received prior to reaching the medical device. According to some embodiments, the method includes analyzing the received instructions in order to detect anomalous instructions before the instructions reach the medical device, thereby potentially preventing anomalous instructions from reaching the medical device.

According to some embodiments, the method includes preprocessing the received instructions. According to some embodiments, the preprocessing may include one or more of cleaning the data of the instructions, encoding categorial features within the instructions, applying standardization, normalizing, and implementing feature selection algorithms. According to some embodiments, the method includes transferring the preprocessed instructions to the first detection layer.

According to some embodiments, at step 104, the method includes analyzing the instructions by applying a first detection layer. According to some embodiments, the first detection layer may be an unsupervised detection layer. According to some embodiments, the first detection layer may include a machine learning model configured to detect and/or identify anomalous instructions. According to some embodiments, the first detection layer may be configured to detect and/or identify context free (CF) anomalous instructions.

According to some embodiments, the method includes identifying context free non-anomalous instructions. According to some embodiments, context free non-anomalous instructions include instructions which have been applied to the first detection layer and were not identified and/or detected as anomalous instructions. According to some embodiments, the method includes transferring the context free non-anomalous instructions from the first detection layer to the second detection layer. According to some embodiments, the second detection layer is configured to receive the context free non-anomalous instructions from the first detection layer. According to some embodiments, the second detection layer is applied to instructions that were not detected and/or identified as anomalous by the first detection layer.

According to some embodiments, at step 106, the method includes analyzing the instructions by applying a second detection layer to the received instructions and/or the context free non-anomalous instructions. According to some embodiments, the second detection layer includes a supervised detection layer. According to some embodiments, the second detection layer includes a supervised machine learning model configured to detect and/or identify anomalous instructions. According to some embodiments, the second detection layer is configured to detect context sensitive (CS) anomalous instructions.

According to some embodiments, and as described in greater detail elsewhere herein, the first detection layer and the second detection layer together are included in a dual-layer architecture. According to some embodiments, the dual-layer architecture includes the preprocessing of the instructions prior to the first detection layer and/or the second detection layer.

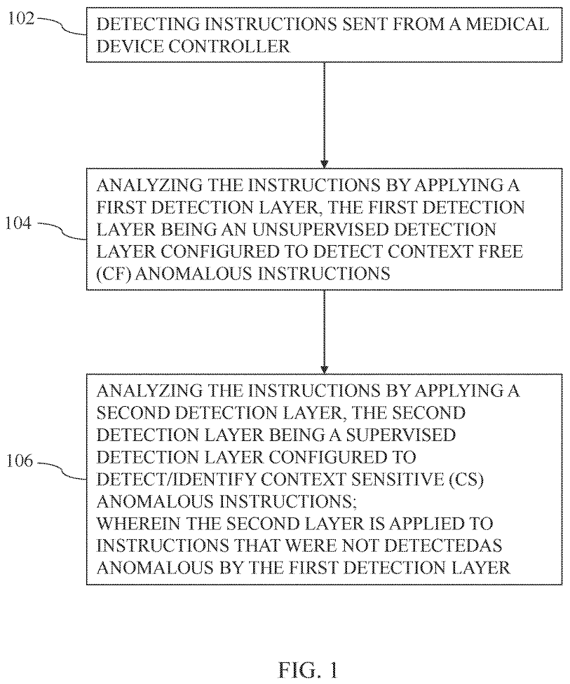

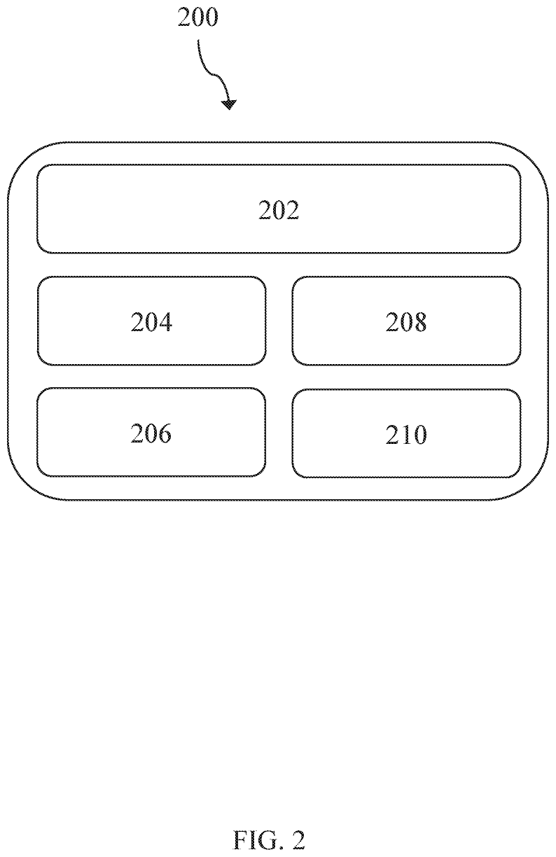

Reference is made to FIG. 2, which shows a schematic illustration of an exemplary device for detection of anomalous instructions, in accordance with some embodiments of the present invention.

According to some embodiments, the device 200 for detection of anomalous instructions is configured to receive instructions sent form the controller of the medical device before the instructions reach the medical device. According to some embodiments, the device 200 may be coupleable to the medical device. According to some embodiments, the device 200 may be in communication with the medical device.

According to some embodiments, the device 200 may be in communication with the controller of the medical device. According to some embodiments, the device can be configured to couple to the controller at a first end thereof and couple to the medical device at a second end thereof. According to some embodiments, the device can be configured to wirelessly communicate with at least one of the controller and the medical device.

According to some embodiments, the device includes a processor 202 configured to receive the instructions from the controller of the medical device. According to some embodiments, the processor 202 is configured to preprocess the received instructions. According to some embodiments, the processor 202 is configured to analyze the received instructions. According to some embodiments, the processor 202 is configured to analyze the received instructions by applying the first detection layer thereto. According to some embodiments, the processor is configured to execute the method for detection of anomalous instructions, as described in greater detail elsewhere herein. According to some embodiments, the first detection layer includes an unsupervised detection layer. According to some embodiments, the first detection layer includes a machine learning model configured to detect context free (CF) anomalous instructions. According to some embodiments, the processor 202 is configured to analyze the received instructions by applying the second detection layer thereto. According to some embodiments, the processor 202 is configured to transfer to the second detection layer only instructions which were not detected as anomalous by the first detection layer. According to some embodiments, the second detection layer includes a supervised detection layer. According to some embodiments, the second detection layer includes a machine learning model configured to detect context sensitive (CS) anomalous instructions. According to some embodiments, the processor 202 is configured to apply the first detection layer and then the second detection layer, in series, to the received instructions. According to some embodiments, the detection of the anomalous instructions may be executed in real time.

According to some embodiments, the device 200 includes a memory module 204 in communication with the processor 202. According to some embodiments, the memory module includes a non-transitory computer-readable medium. According to some embodiments, the memory module includes stored commands. According to some embodiments, the commands stored onto the memory module 204 are configured to cause the processor 202 to detect and/or receive instructions sent from the controller of the medical device, wherein the instruction being may be intended to be received by a medical device. According to some embodiments, the processor is configured to execute the method for detection of anomalous instructions prior to the instructions reaching the medical device. According to some embodiments, the commands stored onto the memory module 204 are configured to cause a processor to analyze the instructions by applying the first detection layer to the received instructions. According to some embodiments, the first detection layer may be an unsupervised detection layer. According to some embodiments, the first detection layer may include a machine learning model. According to some embodiments, the first detection layer may be configured to detect context free (CF) anomalous instructions.

According to some embodiments, the method includes analyzing by applying the first detection layer which includes calculating an anomaly score of the received instructions and comparing the anomaly score with an anomaly threshold. According to some embodiments, the anomaly score is associated with an expected proportion/fraction of anomalies in the instructions, generated during initialization and/or training of the detection layer. According to some embodiments, the first detection layer includes one or more ensemble average algorithms including one or more ensembles. According to some embodiments, the ensemble average algorithm is configured to select an anomaly score using the anomaly scores of the plurality of algorithms of the one or more ensembles. According to some embodiments, applying the first detection layer includes applying a one or more of algorithms (such as, for example, the ensemble algorithms) to the received instructions, wherein each algorithm outputs a score associated with at least one of the received instructions and a potential level of anomaly of the received instructions. According to some embodiments, calculating an anomaly score includes selecting the anomaly score from the scores outputted by the plurality of algorithms associated with the first detection layer.

According to some embodiments, the comparison between the anomaly score and the anomaly threshold is associated with one or more of: a deviation from a predetermined threshold value, a deviation from a corresponding standard parameter value, an unlikely parameter value, and an unlikely combination of parameter value. According to some embodiments, applying the first layer includes determining if one or more parameter values of the received instructions deviate from values of corresponding parameters of a predetermined parameter value data set, wherein a deviation between the one or more parameter values of the received instructions and values of parameters in the predetermined value data set is indicative of the instructions being anomalous.

According to some embodiments, the commands stored onto the memory module 204 are configured to transfer the instructions which were not detected as context free

(CF) anomalous instructions to the second detection layer. According to some embodiments, the commands stored onto the memory module 204 are configured to cause a processor to analyze the instructions by applying a second detection layer. According to some embodiments, the second detection layer may include a supervised detection layer. According to some embodiments, the second detection layer may include a machine learning model. According to some embodiments, the second detection may be configured to detect context sensitive (CS) anomalous instructions.

According to some embodiments, the context sensitive (CS) anomalous instructions relate to one or more context values associated with the received instructions and to a specific patient being potentially and/or intended to be monitored or treated by the medical device by implementing the received instructions. According to some embodiments, the one or more context values related to the specific patient are selected from: type of medical procedure applied to the patient, a selected scan option, region of interest of the patient body being monitored or treated, a study, and a protocol of the instructions. According to some embodiments, the one or more context values related to the specific patient includes characteristics of the specific patient, selected from: age, gender, weight, and medical history.

According to some embodiments, the method includes receiving a context value associated with the received instructions. According to some embodiments, analyzing by applying the second detection layer includes applying the received instructions to at least one supervised classification algorithm configured to output a predicted context value associated with the received instructions. According to some embodiments, analyzing by applying the second detection layer includes comparing the predicted context value with the received context value. According to some embodiments, the second layer is configured to detect the instructions as anomalous if the predicted context value is essentially unequal to the received context value.

According to some embodiments, the device 200 may include a user interface module 206. According to some embodiments, the user interface module 206 may be in communication with the processor 202. According to some embodiments, the user interface module 206 may include one or more of a monitor, a touch screen, a keyboard, a display, an alert unit, and one or more buttons. According to some embodiments, the user interface module 206 may include the Electronic Medical Record (EMR). According to some embodiments, the user interface module 206 is configured to be operated by an operator. According to some embodiments, the operator may be manual. According to some embodiments, the operator may be automated.

According to some embodiments, the device 200 may include and/or be in communication with a database 208. According to some embodiments, the database 208 may be stored within the device 200. According to some embodiments, the database 208 may be stored onto a cloud. According to some embodiments, the processor 202 may be in communication with the database 208. According to some embodiments, the database 208 may include data associated with one or more algorithms of the first detection layer. According to some embodiments, the database 208 may include data associated with one or more algorithms of the second detection layer. According to some embodiments, the database 208 may include data of instructions associated with one or more specified medical devices. According to some embodiments, the database 208 may include data associated with patient profiles. According to some embodiments, the database 208 may include data associated with a history log of operations of a specified medical device.

According to some embodiments, the device 200 may include a protection mechanism 110 configured to protect the data within the device 200 According to some embodiments, the protection mechanism 110 is configured to protect the data during the transfer of the data between the device 200 and at least one of the database 208, the controller, and the medical device. According to some embodiments, the device 200 includes a unidirectional channel coupled to the processor 202 and configured to direct the instructions in only one direction, thereby preventing one or more signals from traveling from the processor 202 to an external device. According to some embodiments, and as described in greater detail elsewhere herein, the protection mechanism 110 may include an out-of-band data mechanism. According to some embodiments, the out-of-band mechanism is configured to inspect the data sent from the controller to the medical device and/or to the processor 202.

According to some embodiments, the out of band data mechanism is configured to inspect the content sent by the controller (or in other words, the host PC) in the form of instructions (e.g., which may be compromised by an adversarial) to the medical device. Advantageously, inspecting the content sent by the controller can be beneficial because the controller may be compromised. According to some embodiments, the out-of-band mechanism includes a channel. According to some embodiments, the channel may be not related, connected and/or coupled, in any way to the medical device, and/or to the compromised device or network. According to some embodiments, the channel is configured to inspect the content sent by the controller in order to prevent damage and/or harm to the subject via the medical device. According to some embodiments, and as described in greater detail elsewhere herein, the out-of-band data mechanism may include one or more one-way links configured to transfer data unidirectionally, thereby adding an additional layer of protection to the data that is being transferred.

According to some embodiments, and as described in greater detail elsewhere herein, the protection mechanism 110 may include a hypervisor algorithm. According to some embodiments, the method includes receiving the instructions using a hypervisor algorithm. According to some embodiments, the hypervisor algorithm is configured to apply the received instructions to a virtual machine (VM). According to some embodiments, and as described in greater detail elsewhere herein, the virtual machine is configured to test the instructions applied to the hypervisor machine.

According to some embodiments, the method includes issuing an alert if anomalous instruction(s) have been identified. According to some embodiments, the device 200 is configured to issue an alert if anomalous instructions have been identified. According to some embodiments, the alert is issued using the user interface module 206.

According to some embodiments, the method includes identifying the detected anomalous instructions as at least one specified type of anomalous instructions. According to some embodiments, identifying the detected anomalous instructions as at least one specified type of anomalous instructions is based, at least in part, on an irregularity identified by at least one of the first detection layer and said second detection layer. According to some embodiments, identifying the detected anomalous instructions as at least one specified type of anomalous instructions includes, at least in part, classifying an irregularity identified by at least one of the first detection layer and said second detection layer into at least one irregularity type classification. According to some embodiments, the method includes assigning a risk score to the detected anomalous instructions associated with a severity level of the detected anomalous instructions. According to some embodiments, the risk score is based, at least in part, on the irregularity type classification of the detected anomalous instruction.

According to some embodiments, the method includes detecting an error associated with the generation of the received instructions from the controller using one or more values associated with the detected anomalous instructions, and wherein said generation is automatic and/or manual. According to some embodiments, the method includes preventing or blocking a detected anomalous instruction from reaching the medical device. According to some embodiments, the method includes generating remediation instructions configured to replace the anomalous instructions. According to some embodiments, the method includes outputting recommended instructions based, at least in part, on at least one of the detected anomalous instructions and the type of anomalous instructions. According to some embodiments, the method includes outputting a signal to the medical device including instructions based, at least in part, on at least one of the detected anomalous instructions and the type of anomalous instructions, wherein the signal is associated with remediation instructions configured to replace the anomalous instructions.

### Dual Layer Structure

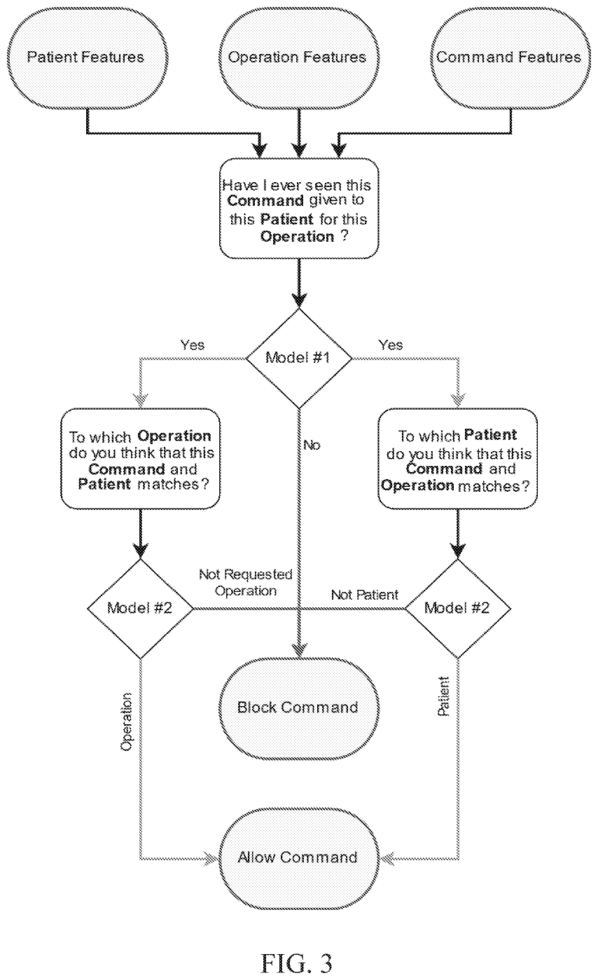

According to some embodiments, the dual layer architecture is configured to detect anomalies of instructions with extreme values (such as, e.g., ×100 more radiation than normal). According to some embodiments, the dual layer architecture is configured to detect anomalies of normal instructions'values that were sent to a wrong subject (such as, e.g., a normal instruction for a person weighing 100 kg sent to an infant weighing 10 kg). According to some embodiments, each type of anomaly requires a different detection approach, and therefore the anomaly detection algorithm includes two layers. Reference is made to FIG. 3, which shows a schematic illustration of an exemplary system for detection and prevention of malicious instructions, in accordance with some embodiments of the present invention. According to some embodiments, the system is configured to detect of malicious commands based, at least in part, on a plurality of features inputted into the system. According to some embodiments, the features include at least one, at least two, or at least three types of features. According to some embodiments, the types of features may include one or more of a patient related feature, an operation related feature, and an instruction related feature.

According to some embodiments, the features may include a patient related feature, such as, for example, the age, gender, weight, height, of the subject. According to some embodiments, the features may include an operation related feature, such as, for example, abdomen CT scan, head CT scan, and chest CT scan. According to some embodiments, the features may include an instruction related feature.

According to some embodiments, similar patient profiles are considered instead of individual patients. According to some embodiments, some patients share many common features (i.e., age), and therefore similar patient profiles may be considered rather than individual patients. According to some embodiments, operation related features may be divided into sub-types, rather than individual operations. According to some embodiments, operation related features may be divided into individual operations. According to some embodiments, operation related features may be divided into sub-types which may then be divided into sub-sub-types and/or individual operations.

According to some embodiments, the instruction related features may be regarded individually. According to some embodiments, the instruction related features may require pre-processing in order to export relevant features.

According to some embodiments, the types of features may include one or more of a radiation level, size of the area being scanned/irradiated, the position of the mechanical bed used with the medical device, the velocity of rotating motors of the medical device, and the sensors configuration of the medical device.

According to some embodiments, the dual layer architecture is configured to receive instruction from the controller of the medical device. According to some embodiments, dual layer architecture is configured to receive the features. According to some embodiments, the first detection layer is configured to receive the features. According to some embodiments, the second detection layer is configured to receive the features from the first detection layer. According to some embodiments, the system is configured to output an alert if any layer detects an anomaly instruction. According to some embodiments, the dual layer architecture is configured to receive the instructions before the instructions reach the medical device. According to some embodiments, the system is configured to entirely prevent the anomaly instructions from reaching the medical device and affecting the patient, since the instructions have not yet reached the medical device. According to some embodiments, the origin of the anomaly does not affect the efficiency of detection layers. According to some embodiments, it does not matter where the anomaly was initiated from (i.e., the specific malfunction device) since eventually, the anomaly instruction must pass through the system and thus will be detected, thereby making the system and/or device provided herein a solution which can detect and prevent many different anomalies.

According to some embodiments, the first detection layer is configured to detect instructions which include anomalies having instructions with extreme values. According to some embodiments, the first detection layer is configured to identify the instructions. According to some embodiments, the first detection layer is configured to identify if the instructions is similar to a previous and/or a known instruction. For example, in some embodiments, the first detection layer is configured to compare the instructions to a known database of instructions and/or to a log of previously received instructions of previously received instructions. According to some embodiments, the first detection layer is configured to identify if the received instructions and/or similar previously received instructions have been given to a same and/or similar patients and/or patient having a similar profile. According to some embodiments, the first detection layer is configured to identify if the received instructions and/or similar previously received instructions have been given to a same and/or similar patients and/or patient having a similar profile for a similar type of scan and/or operation. In other words, according to some embodiments, the first detection layer is configured to answer the question: “have I ever seen this instruction, given to this type of patient, for this requested type of scan?”.

A potential advantage of the first detection layer being configured to identify the received instructions by identifying if the type of instruction has been received for similar subject and/or for similar operations is in that the first layer is configured to detect anomalies which cannot be detected by placing a rule-based system and/or a threshold for operational parameters, and/or having predefined rules (such as, e.g., a threshold radiation value of no more than X). According to some embodiments, many parameters of the instructions cannot be limited by a threshold, as some procedures require certain high values for parameters. According to some embodiments, medical devices are commonly carefully tested as part of the rigorous regulations for safety properties. According to some embodiments, some medical devices already implement such a rule-based system, and there is a certain allowed gap for the instructions' values. According to some embodiments, the first detection layer and/or the second detection layer are configured to detect anomalies within the allowed gap of the medical devices that may still cause damage if misused.

According to some embodiments, the first detection layer is configured to detect extreme anomalies within the received instructions. According to some embodiments the first detection layer is configured to analyze the instructions while taking into account some distribution of the parameters of the instructions and use a statistical-based anomaly detection, thereby detecting anomalies in the instructions (for example, e.g., an extreme amount of radiation).

According to some embodiments, the first detection layer may include one or more classification methods and/or algorithms, such as, for example, support vector machine (SVM). According to some embodiments, the classification methods and/or algorithm may be used for cases of extreme instructions, or in other words, instructions which have values that are considered unusual and/or irregular in relation to normal instructions, and/or unusual and/or irregular in relation to commonly received instructions. According to some embodiments, the first detection layer is configured to reduce the dimensionality of the received instructions. According to some embodiments, the first detection layer is configured to reduce the dimensionality of the received instructions using spectral based anomaly detection. According to some embodiments, the received instructions may include numerous parameters (and in some embodiments may include hundreds of parameters). According to some embodiments, the anomaly within the received instructions may be related to only one, or a few, or a plurality of the parameters within the received instructions. Advantageously, reducing the dimensionality of the received the instructions allows identifying the anomaly within the received the instructions by identifying the specific parameters related to the anomaly of the anomalous instructions.

According to some embodiments, the first detection layer is configured to send the instructions which were not identified as anomalous to the second detection layer. According to some embodiments, the second detection layer is configured to receive the instructions from the first detection layer, wherein the instructions received by the second detection layer were identified as non-anomalous by the first detection layer.

According to some embodiments, the second detection layer is configured to detect if an instruction sent by the controller may be anomalous in that it is considered a mistake to send such an instruction to a specific subject and/or to a specific medical device. According to some embodiments, the second detection layer is configured to detect anomalous instructions relating to instructions sent by the controller for the wrong patient or operation. According to some embodiments, the second detection layer can assume that the received instructions does not contain extreme values or values never seen before, because such instructions would have been detected and screened by the first detection layer. According to some embodiments, the instructions received by the second layer must match some patient (such as, i.e., a patient profile) for some operation (such as, i.e., an operation type).

According to some embodiments, the second detection layer is configured to identify an operation which matches with the received instructions. According to some embodiments, the second detection layer is configured to identify an operation which matches with the received instructions in relation to the patient profile. According to some embodiments, the second detection layer is configured to answer the question “to which type of operation does this instructions and patient profile matches?”.

According to some embodiments, the second detection layer is configured to compare the identified patient profile with the real and/or the received operation type and output an alert if they do not match. According to some embodiments, the second detection layer is configured to compare the identified patient profile with the real and/or the received operation type and detect the instruction as anomalous if the identified patient profile and the real patient profile do not mach.

According to some embodiments, the second detection layer is configured to identify a patient profile which matches with the received instructions. According to some embodiments, the second detection layer is configured to identify a patient profile which matches with the received instructions in relation to the operation. According to some embodiments, the second detection layer is configured to answer the question “to which patient profile does this instructions and operation type matches?”.

According to some embodiments, the second detection layer is configured to compare the identified operation type with the real and/or the received operation type and output an alert if they do not match. According to some embodiments, the second detection layer is configured to compare the identified operation type with the real and/or the operation type and detect the instructions as anomalous if the identified patient profile and the real patient profile do not mach.

For example, in some embodiments, if an infant patient is performing operation of abdomen CT scan, and the algorithm predicted that the sent instructions matches an adult, the second detection layer will output an alert.

According to some embodiments, the second detection layer is configured to identify a patient profile, identify an operation type, or identify both a patient profile and an operation type. According to some embodiments, the second detection layer is configured to use a combination of the identified patient profile, the identified operation type, the compared patient profiles, and the compared operation types, in order to identify if an instruction is anomalous.

For example, a classification algorithm of the second detection layer can classify instructions to different groups of patients and/or operation types, and therefore multi-class classification and/or clustering-based anomaly detection can be implemented.

### Anomaly Detection

According to some embodiments, the dual layer architecture is configured to detect and/or identify anomalous instructions using at least one of the first detection layer and the second detection layer. According to some embodiments, the dual layer architecture includes at least one machine learning model in at least one of the layers. According to some embodiments, the layers of the architecture are configured to be implemented in series.

According to some embodiments, the instructions received/detected by the dual layer architecture may include one or more signal sent from the controller and configured to be received and/or implemented by the medical device. According to some embodiments, the instructions may include a set of signals.

According to some embodiments, and as described in greater detail elsewhere herein, the instructions may be considered anomalous for having one or more doubtable values. According to some embodiments, the dual layer architecture is configured to identify one or more instructions as anomalous if the instructions include one or more signal that is identified as abnormal, unexpected, and/or unusual. According to some embodiments, the dual layer architecture is configured to identify one or more instructions as anomalous if the instructions include one or more signal that is identified as abnormal in relation to an existing database of data relating to operation of the medical device. According to some embodiments, the dual layer architecture is configured to identify one or more instructions as anomalous if the instructions include one or more signal that is identified as abnormal in relation to an existing database of data relating to medical treatment of the specific subject being treated using the medical device.

According to some embodiments, the first detection layer and the second detection layer are configured to identify the anomalous instructions. According to some embodiments, the first detection layer and the second detection layer are configured to identify the anomalous instructions based, at least in part, on different databases of data regarding at least one of the medical devices and the subject being treated by the medical device.

According to some embodiments, the first detection layer and the second detection layer include different machine learning models configured to detect the anomalous instructions. According to some embodiments, the first detection layer and the second detection layer may include one or more supervised machine learning models and/or unsupervised machine learning models. According to some embodiments, the supervised machine learning models may include one or more of decision trees, k-Nearest Neighbors (k-NN), and Multilayer Perceptron (MLP) neural network. According to some embodiments, the unsupervised machine learning models may include one or more of One Class Support Vector Machine (OCSVM), and AutoEncoder (AE)).

According to some embodiments, the dual layer architecture is configured to identify context-free (CF) anomalous instructions. According to some embodiments, context-free anomalous instructions may include instructions which do not relate to a specific individual subject that is being treated by the medical device. e.g., unlikely values or combinations of values, of instruction parameters (e.g., giving 100 times more radiation than usual); and (2) Context-sensitive (CS) anomalous instructions (e.g., normal values or combinations of values, of instruction parameters that are considered anomalous within a particular context (e.g., a wrong scan type, or mismatching patient age).

According to some embodiments, some anomalies are context sensitive and are only considered anomalous given a specific context.

According to some embodiments, the medical device is configured to image one or more portions of the body of the subject. According to some embodiments, the medical device includes an imaging device, such as, for example, computed tomography (CT), magnetic resonance imaging (MRI), radiography (X-ray machine), and Positron emission tomography (PET).

In some embodiments, the medical device is configured to produce an imaging scan, or in other words, a series, that includes a sequence of images (slices). According to some embodiments, the sequence of images may include two-dimensional images and/or three-dimensional images. According to some embodiments, the instructions sent by the controller include data relating to at least one of the series and the slices. According to some embodiments, the instructions relate to the medical device operational parameters for implementation of the imaging of the subject. According to some embodiments, the medical device operational parameters include the scan options of the medical device.

According to some embodiments, the scan options may include axial slices (in which the slices are along/parallel the z-axes), helix slices (in which the series of slices produce a helical shape along a portion of the body of the subject, like a screw), and surview slices (which includes an initial brief scan, with very low radiation, that allows the operator to configure the subsequent scans better, as well as apply various optimization techniques).

According to some embodiments, the clinical procedure of a scan, such as a CT scan, is called a Study. According to some embodiments, the medical device operator does not configure the Study one scan at a time. According to some embodiments, specific sequences of medical device scans are predefined as a set of Protocols from which the operator can choose from. According to some embodiments, a single Study can combine more than one Protocol (e.g., a Chest/Abdomen Study combines Chest Protocol and Abdomen Protocol). According to some embodiments, the Study usually depends on the Body Part being scanned. According to some embodiments, the Scan Options, Body Part, Study, and Protocol may be individual and/or different (abstractions) of clinical objective contexts.

According to some embodiments, the dual layer architecture is configured to classify the clinical objective context (abstractions) within a set of classes. According to some embodiments, an analysis of collected instructions revealed that the clinical objective context (abstractions) uses a predefined finite set of classes. According to some embodiments, the classes include a hierarchical relationship. According to some embodiments, each of the clinical objective context (abstractions) include a plurality of classes into which the clinical objective context (abstractions) may be classified to.

For example, according to some embodiments, the Scan Option includes 3 classes into which the Scan Options may be classified. According to some embodiments, the Body Part of the subject that is being scanned can be classified into 11 classes. According to some embodiments, the Study can be classified into 34 classes. According to some embodiments, the Protocol can be classified into 72 classes.

A potential advantage of having different classifications for different clinical objective context (abstractions) is in that a deeper hierarchy level provides more information about the clinical objective.

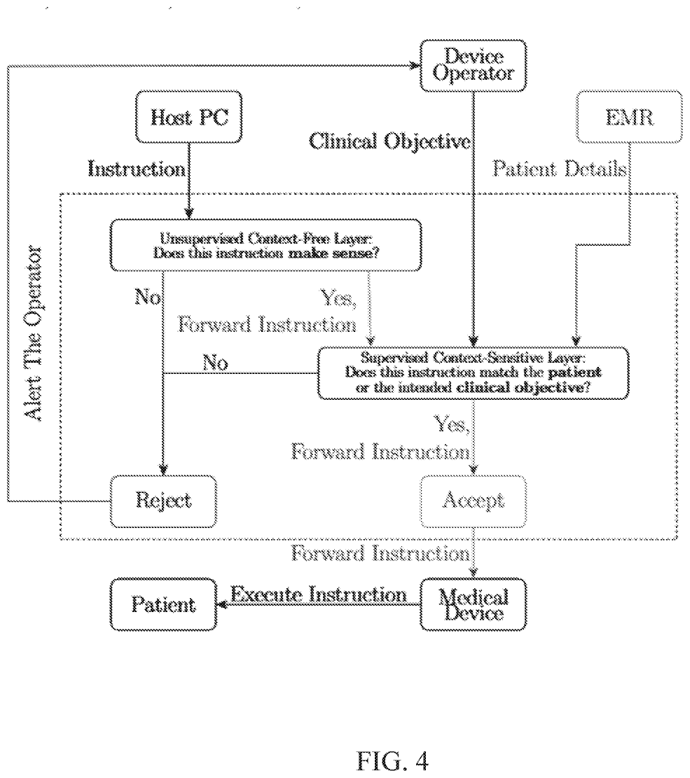

Reference is made to FIG. 4, which is a schematic illustration of a dual-layer architecture for the protection of medical devices from anomalous instructions, using both the context-free (CF) and the context-sensitive (CS) layers, in accordance with some embodiments of the present invention. According to some embodiments, the dual-layer architecture can be designed to detect anomalous instructions using two algorithmic layers (a CF one and a CS one). According to some embodiments, the dual-layer architecture can be designed to implement and/or evaluate anomalous instructions using two algorithmic layers (a CF one and a CS one). According to some embodiments, the implementation of each layer includes using a set of specific classifiers.

According to some embodiments, the dual-layer architecture for the protection of medical devices can be evaluated using instructions from a controller of the medical device, and/or by focusing on detection of clinical objective CS anomalous instructions. According to some embodiments, it is possible to implement the architecture using other classifiers and evaluate it on different medical devices.

According to some embodiments, at least one layer includes a database and/or is trained using a database. According to some embodiments, the database includes data relating to a specified medical device. According to some embodiments, the database is based, at least in part, on records of instructions of a type medical device, similar to or same as the specified medical device.

According to some embodiments, the database is based, at least in part, on data collected from one or more medical devices. According to some embodiments, the data is collected using a data collection tool. According to some embodiments, the data collection tool is configured to record instructions sent from the host PC to the gantry (for example, e.g., the physical component of the CT). For example, according to some embodiments, in order to collect real data, the data collection tool can be installed in CT scanners. According to some embodiments, the collected data can include the instruction parameters (which may include, for example, 233 features) and/or instruction metadata (which may include, for example, 77 features). According to some embodiments, the metadata is logged by the host PC but is not part of the instruction parameters and is not sent to the gantry. According to some embodiments, the metadata includes clinical objective context (for example, e.g., Scan Options, Body Part, Study, and/or Protocol), and/or patient context (for example, e.g., gender and/or age).

According to some embodiments, at least a portion of the dual-layer architecture is trained on a train set of instructions. According to some embodiments, the train set includes the collected data. According to some embodiments, the collected data is separated into a train set and a test set. According to some embodiments, the train set includes 75% of the data regarding instructions sent to the medical device. According to some embodiments, the test set includes 25% of the data regarding instructions sent to the medical device.

According to some embodiments, the instructions in the train set include one or more labels associated with context free anomalous instructions. According to some embodiments, the labels of the instructions are labeled by a professional/expert, such as an expert operator. According to some embodiments, the context sensitive anomalous instructions include repetitions of a same or similar procedure for a same subject.

According to some embodiments, the received instructions are pre-processed before being analyzed by the dual-layer structure. According to some embodiments, the pre-processing includes cleaning the data. According to some embodiments, cleaning the data includes removing instructions that include one or more parameters with Not a Number (NaN) values. According to some embodiments, the preprocessing includes encoding categorical features. According to some embodiments, the preprocessing includes applying standardization, such as, for example, Z-score normalization. According to some embodiments, the preprocessing includes implementing feature selection algorithms, for example, in order to drop features with a single value and/or features with a 100% correlation with other features. According to some embodiments, for the supervised second detection layer, each of the labels in the train set included at least 100 examples of anomalous instructions.

According to some embodiments, the method includes receiving from the controller and/or the host PC. According to some embodiments, the method includes receiving at least one clinical objective and/or patient-specific context from the operator and/or the Electronic Medical Record (EMR). According to some embodiments, and for security reasons, the context can be sent from an isolated secure private channel and not directly from the controller and/or host PC. A potential advantage of sending the context from an isolated secure private channel is in that otherwise, a compromised host PC may send a malicious context matching the anomalous instructions.

According to some embodiments, the first detection layer is configured to detect context-free (CF) anomalous instructions. According to some embodiments, the first detection layer is configured to receive the instructions as input. According to some embodiments, the first detection layer includes a pre-trained unsupervised anomaly detection algorithm to detect CF anomalous instructions. According to some embodiments, the pre-trained unsupervised anomaly detection algorithm is trained using non-anomalous instructions from one or more databases of data regarding specified medical device. According to some embodiments, the first detection layer is configured to output an alert if an anomalous instruction is detected. According to some embodiments, the first detection layer is configured to send the instructions that were not detected as anomalous to the second detection layer.

According to some embodiments, the first detection layer includes a plurality of unsupervised anomaly detection algorithms. According to some embodiments, each algorithm is configured to calculate an anomaly score of a received instruction. According to some embodiments, the first detection layer is configured to compare the anomaly score to an anomaly threshold. According to some embodiments, instructions for which the anomaly score is above the anomaly threshold, the instructions are detected as anomalous. According to some embodiments, the anomaly threshold is calculated as the 100·(1−contamination) percentile of the anomaly scores of the training set, wherein contamination relates to the expected proportion of anomalies in the data. According to some embodiments, the contamination is calculated during the training of the one or more algorithms. According to some embodiments, the contamination is calculated and/or determined during the initialization of the algorithms.

According to some embodiments, the first detection layer includes one or more ensemble average algorithms. According to some embodiments, the first detection layer and/or the ensemble average algorithm includes one or more ensembles, each including a plurality of algorithms, for example, such as Angle-based Outlier Detector(ABOD), k-NN, One-Class Support Vector Machine (OCSVM), and Isolation Forest (IForest).

According to some embodiments, the first detection layer and/or the ensemble average algorithm is configured to select an anomaly score based, at least in part, on the anomaly scores of the plurality of algorithms of the one or more ensembles. According to some embodiments, the first detection layer and/or the ensemble average algorithm is configured to select an anomaly score using the anomaly scores of the plurality of algorithms of the one or more ensembles. According to some embodiments, the first detection layer and/or the ensemble average algorithm implements a Locally Selective Combination of Parallel Outlier Ensembles (LSCP) to choose an anomaly score of the one or more ensembles.

According to some embodiments, the selected anomaly score of the ensemble includes the maximal anomaly score of the algorithms of the ensemble. According to some embodiments, the selected anomaly score of the ensemble includes the average anomaly score of the algorithms of the ensemble. According to some embodiments, the final anomaly score of the ensemble is compared to the anomaly threshold of the specific ensemble.

A potential advantage of the architecture having a second detection layer is in that instructions that were not detected as anomalous, which might still be context sensitive anomalous instructions within a particular context, can be detected using the second detection layer.

According to some embodiments, adding the second detection layer to the architecture improves the overall anomaly detection performance from an F1 score of 71.6% (using only the first detection layer) to 82.3%-98.8%. According to some embodiments, the second detection layer enables the detection of Context Sensitive anomalies, using the semantics of the device's procedure, which cannot be detected using only the first detection layer, which may be purely syntactic.

According to some embodiments, the contamination parameter ranges between 0.001-0.02. According to some embodiments, the contamination parameter is essentially 0.01. According to some embodiments, the algorithms of the dual layer architecture are trained using non-anomalous instructions

According to some embodiments, the second detection layer is configured to receives the instructions from the first detection layer. According to some embodiments, the second detection layer is configured to receive data associated with the intended instruction contexts. According to some embodiments, the data associated with the intended instruction contexts includes any one or more of clinical objective (e.g., which may be provided by the technician operating the device) and one or more patient characteristics (for example, such as, characteristics that are provided by the EMR). According to some embodiments, the second detection layer uses one or more pre-trained set of supervised classification algorithms to predict the contexts of the instructions. According to some embodiments, the second detection layer includes a plurality of multi-class classification algorithms for the detection of context sensitive anomalous instructions. According to some embodiments, the multi-class classification algorithms may include one or more of a Decision Tree (DT), Gradient Boosting (GB), k-NN, MLP, and Random Forest (RF).

According to some embodiments, the received instruction context is used as target labels for the one or more supervised classification algorithms. According to some embodiments, the second detection layer is configured to compare the predicted contexts to the received instruction contexts. According to some embodiments, the second detection layer is configured to detect an instruction as anomalous if the predicted contexts do not match with the received instruction contexts.

According to some embodiments, the algorithms are trained using non-anomalous labeled instructions. According to some embodiments, the algorithms are using a test set which does not include anomalous instructions. According to some embodiments, the comparison between the predictions of a context sensitive instruction and the received instruction contexts should be true for non-anomalous instructions and false for context sensitive anomalous instructions.

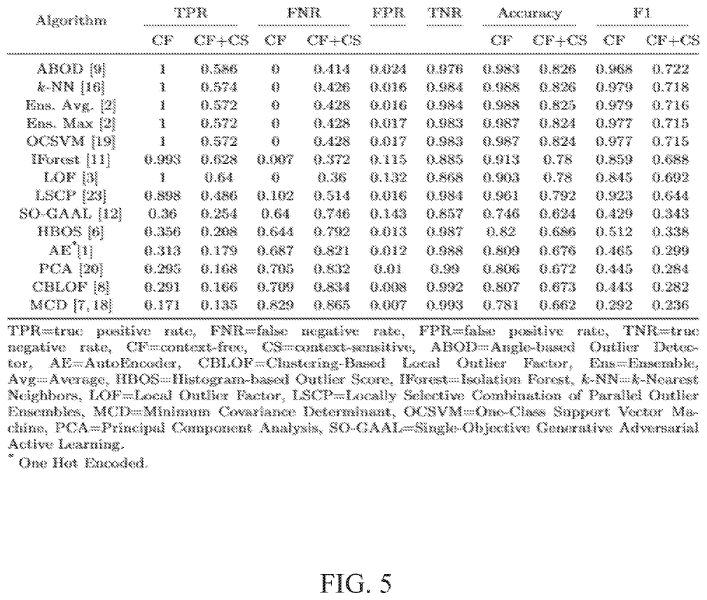

Reference is now made to FIG. 5, which is a table of exemplary results of the unsupervised anomaly detection for the first detection layer, in accordance with some embodiments of the present invention. According to some embodiments, such as depicted in FIG. 5, the performance on the context free anomalous instructions are high for several algorithms, and the highest performance is performed by the Ensemble Average algorithm. According to some embodiments, such as depicted in FIG. 5, the performance on the context free and context sensitive anomalous instructions are much lower, since the algorithms fail to detect the context sensitive anomalous instructions. According to some embodiments, such as depicted by FIG. 5, the number of instructions that were used is lower than the number of collected instructions due to the preprocessing, for example, Surview type instructions were removed, as these types of instructions are quick initial scans that have a lower potential of damage.

According to some embodiments, the second detection layer receives instructions that were detected as non-context-free anomalous by the first detection layer, since the context free anomalous instructions are already detected by the first detection layer.

According to some embodiments, the clinical objective is represent using the four hierarchical abstractions of the scan type. According to some embodiments, the clinical objective is used as the target labels of the supervised classification algorithms for training of the algorithms.

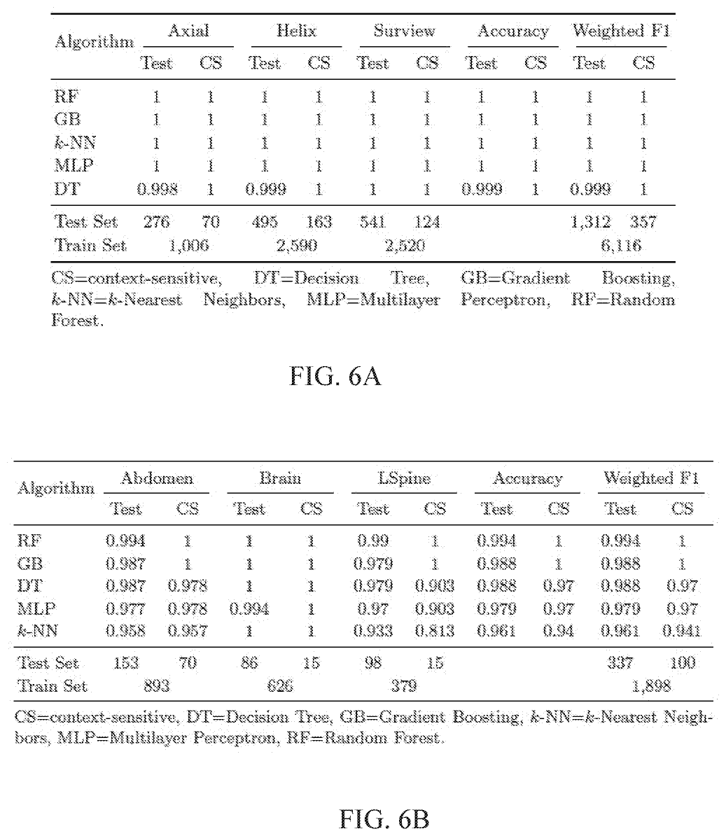

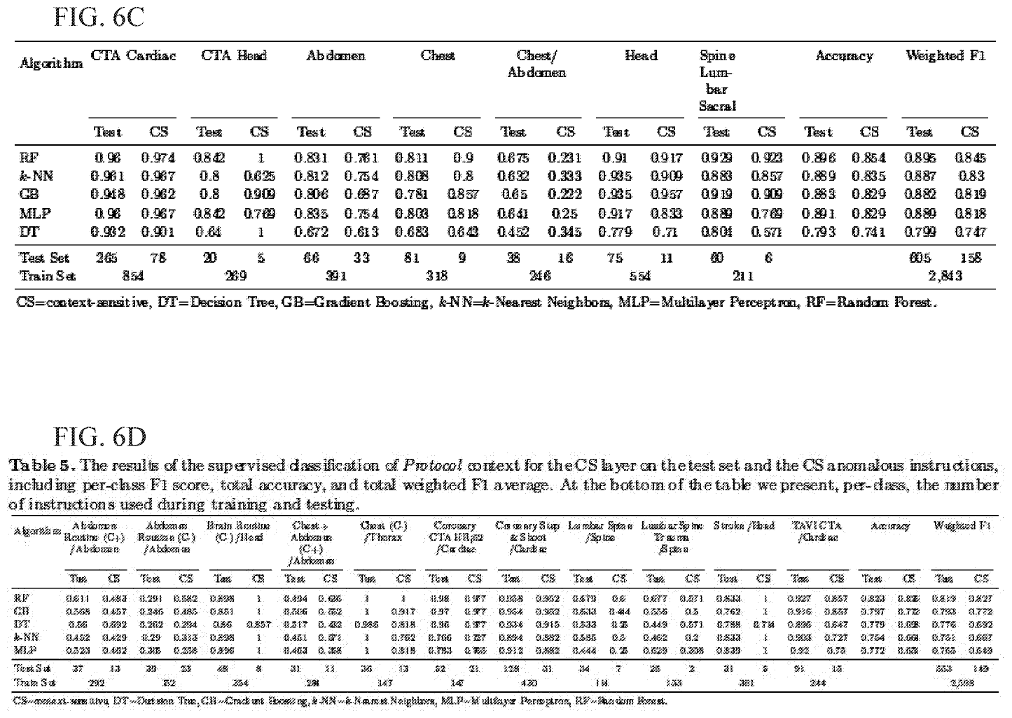

Reference is made to FIG. 6A, FIG. 6B, FIG. 6C, and FIG. 6D, which show tables of exemplary results of the dual-layer architecture, showing the performances of the first detection layer alone and with the additional second detection layers, in accordance with some embodiments of the present invention. According to some embodiments, such as depicted by FIG. 6A, FIG. 6B, FIG. 6C, and FIG. 6D, and as described in greater detail elsewhere herein, the first detection layer includes the ensemble average algorithms. According to some embodiments, such as depicted by FIG. 6A, FIG. 6B, FIG. 6C, and FIG. 6D, the second detection layer includes an RF classifier.

FIG. 6A shows an exemplary algorithm using supervised classification for the Scan Option context objective, in which most of the algorithms reached an F1 score of 1. According to some embodiments, the Scan Option context objective is the highest level of the hierarchy of the context objectives. Thus, in such embodiments, the topmost level of the scan type hierarchical separates instructions very well, for example, in the exemplary algorithms shown in FIG. 6A. According to some embodiments, for the classification process, the Surview instructions may not be removed during the preprocessing.

FIG. 6B shows an exemplary algorithm using supervised classification for the Body Part context objective, in which a portion of the algorithms reach an F1 score of 1. According to some embodiments, the Body Part context objective is second after the Scan Option context objective in the hierarchy. According to some embodiments, the number of total instructions is lower in the second level of hierarchy, such as depicted in FIG. 6B, since not all instructions include the Body Part label. According to some embodiments, the instructions including Surview Scan Option may be removed for the second hierarchy level.

FIG. 6C shows an exemplary algorithm using the supervised classification algorithms for a third level (lower level) context objective, the Study Objective, in which the F1 score decreased in comparison with the higher-level abstractions as seen in FIG. 6A and FIG. 6B.

FIG. 6C shows an exemplary algorithm using the supervised classification algorithms for a fourth level (lowest level) context objective, the Protocol Objective, in which the F1 score decreased in comparison with higher-level abstractions (or higher-level context objectives).

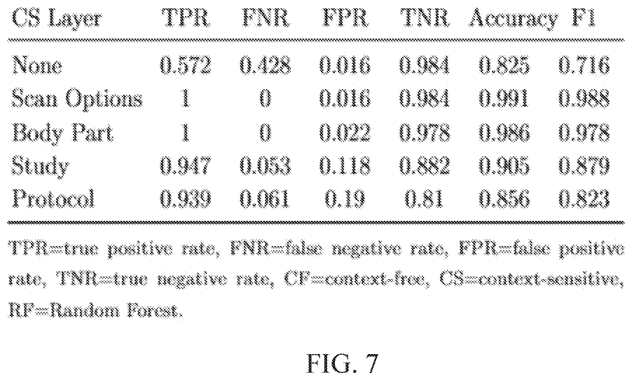

Reference is made to FIG. 7, which shows exemplary results of the dual-layer architecture, showing the performance of the first detection layer alone, and with the additional second detection layers, in accordance with some embodiments of the present invention. According to some embodiments, such as depicted in FIG. 7, and as explained in greater detail elsewhere herein, the first detection layer includes that ensemble average algorithms. According to some embodiments, such as depicted in FIG. 7, the second detection layer includes a RF classifier.

According to some embodiments, such as depicted in FIG. 7, using only the first detection layer led to detecting only 57.2% of the anomalous instructions, while adding the second detection layer and/or knowledge of the Study clinical objective led to a sensitivity of 94.7%.

According to some embodiments, the sensitivity of the dual layer structure ranges between 90% to 99%. According to some embodiments, the sensitivity of the dual layer structure ranges between 93% to 97%.

According to some embodiments, the second detection layer is configured to detect 82%-100% of the context sensitive anomalous instructions. According to some embodiments, the second detection layer is configured to detect context sensitive anomalous instructions by comparing supervised classification methods' predictions (e.g., RF) to the received instructions context. According to some embodiments, the received instructions context which may be received from a separated secure private channel.

According to some embodiments, a dual layer structure having a second detection layer may result in the overall F1 score ranging between 90%-98.8%. According to some embodiments, the first detection layer without the second detection layer may result in the overall F1 score ranging around 71.6%. According to some embodiments, the first detection layer without the second detection layer may result in the overall F1 score ranging between 65%-73%.

### Protective Mechanism—Out of Band

According to some embodiments, there is provided a protection device and/or system for protecting a medical device. According to some embodiments, the protection device and/or system includes a protective layer mechanism. According to some embodiments, the protective mechanism includes an out-of-band data mechanism.

According to some embodiments, the out-of-band data mechanism is configured for detecting and/or preventing anomalous instructions.

According to some embodiments, the out of band data mechanism is configured to inspect the content sent by the controller (or the host PC) in the form of instructions (e.g., which may be compromised by an adversarial) to a medical device. According to some embodiments, the controller may be compromised, thereby the instructions sent by the controller may need to be inspected. According to some embodiments, using an out-of-band mechanism includes an additional channel, which is not related (i.e., such as, connected or coupled) in any way to the compromised device or network. According to some embodiments, the channel is configured to inspect the content sent by the controller in order to prevent damage and/or harm to the subject via the medical device.

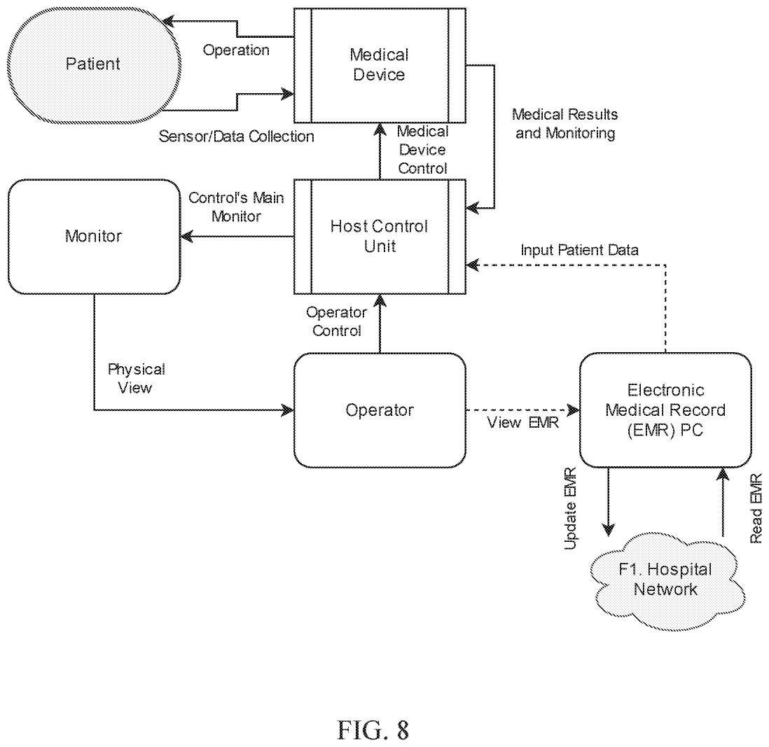

Reference is made to FIG. 8, which shows a schematic data-flow diagram illustration of a medical device unit with a controller, according to some embodiments of the present invention.

According to some embodiments, such as depicted in FIG. 8, there is an interaction between the patient and the medical device. According to some embodiments, the interaction involves one or more operations such as, e.g., a CT scan which emits radiation thereby resulting in an output image. According to some embodiments, the medical device unit includes a medical device in communication with a controller (such as, for example, the host control unit as depicted in FIG. 8). According to some embodiments, the controller may be an integral portion of the medical device unit. According to some embodiments, the medical device is configured to receive instructions from the controller. According to some embodiments, the controller is configured to be a central command unit of the medical device.

According to some embodiments, the medical device unit includes one or more monitor in communication with the controller. According to some embodiments, the monitor includes a screen and may be configured to present information about the operation to an operator. According to some embodiments, the medical device unit includes an operator unit, such as the operator depicted by FIG. 8, configured to input operational data into the operator unit. According to some embodiments, the operator unit may include an operational software. According to some embodiments, the operator may be a person whom inputs data into the operator unit. According to some embodiments, the operator is configured to send instructions to the medical device via the host control unit. Note that the operator might be a person or software.

According to some embodiments, the out-of-band data mechanism is configured to protect the medical device. According to some embodiments, the out-of-band mechanism is configured to protect the endpoint to which the final instructions reach, such as, for example, the medical device. According to some embodiments, the out-of-band mechanism is configured to protect the medical device and/or the endpoint from the outside environment such as, for example, the hospital's network.

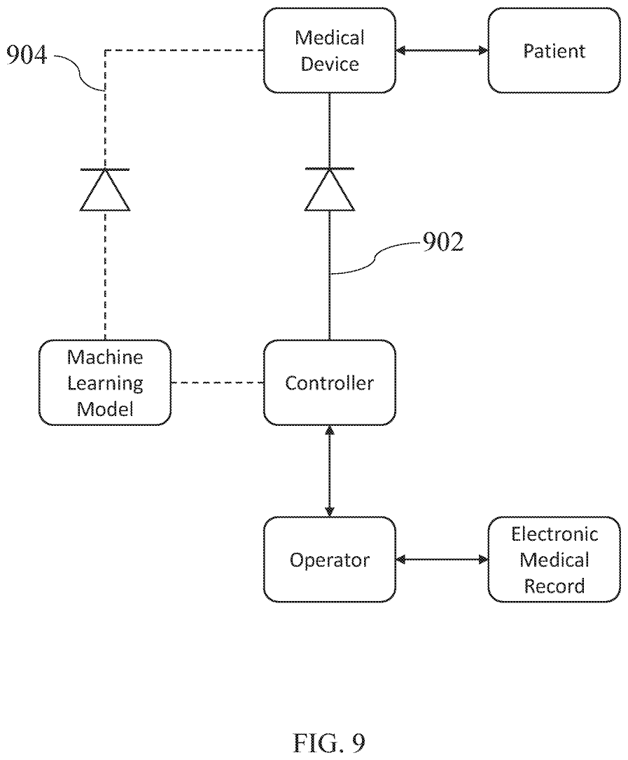

Reference is made to FIG. 9, which shows an exemplary schematic illustration of an out-of-band channel, in accordance with some embodiments of the present invention.

According to some embodiments, the out-of-band channel is configured for the detection and/or prevention framework of anomalous instructions sent from an untrusted source. According to some embodiments, the out-of-band channel is configured to be placed between the controller, such as the host control unit, and the controlled medical device. According to some embodiments, the out-of-band channel is configured to analyze the instructions that the controller sends to the medical device. According to some embodiments, the out-of-band channel is configured to secure the medical device unit such that the medical device unit is protected from adversarial.

According to some embodiments, the physical properties of standard computer connections allow data to flow either inwards and outwards of the connection (e.g., Ethernet, Wi-Fi); thus, it is possible to attack the detection and prevention framework (e.g., using a potential vulnerability). According to some embodiments, in order to avoid the potential vulnerability, a specific computer connection is used. According to some embodiments, the specific computer connection includes a one-way link.

According to some embodiments, one-way links are built physically differently from traditional cables, by making use of light as a means of information transfer rather than electricity. Since light requires a source and a detector, if only one source and one detector are installed, it is possible to guaranty that data will only pass from the source to the detector (i.e., one-way), and not vice-versa. According to some embodiments, the one-way data transfer is guaranteed by the physical properties of the channel (i.e., the cable), thereby allowing the cable resilience to any future vulnerabilities that may be found, which may require two-way connection (e.g., a malware that communicates with remote server, a typical scenario).

According to some embodiments, the out-of-band data mechanism includes a one-way link. According to some embodiments, the one-way link is configured to create a one-way data transfer channel from the source (such as the controller) and the detector (such as the dual layer architecture).

According to some embodiments, the out-of-band data mechanism includes two or more one-way links. According to some embodiments, the out-of-band data mechanism includes two one-way links.

According to some embodiments, the two one-way links are configured to provide an additional layer of protection to the device and/or system. According to some embodiments, the characteristics of the communication between the controller and the medical device enables the use of one-way links (i.e., only being able to send instructions from the controller to the medical device and not receive a reply). According to some embodiments, the two one-way links are coupled to the device such that the device is surrounded by the two one-way links. For example, in some embodiments, the data transfer direction is with respect to the direction of the diode as depicted in FIG. 9.

Advantageously, surrounding the device and/or system with the two one-way links allows the system to be extremely hard to attack remotely (e.g., via the host), which would be the case if an attacker attempts to bypass it and send malicious instructions to the medical device directly.

According to some embodiments, such as depicted in FIG. 9, the one-way link may include a diode. According to some embodiments, the one-way link may be coupled to the controller on one end thereof and to the medical device at a second end thereof, such as, for example, but not limited to, as depicted by connection 902 in FIG. 9. According to some embodiments, the machine learning model and/or the dual layer architecture may be in communication with the controller and/or the medical device.

According to some embodiments, such as depicted by connection 904 of FIG. 9, the machine learning module may be in communication with the medical device via one or more one-way links.

According to some embodiments, such as depicted in FIG. 9, the dual layer mechanism is configured to couple to the controller and the medical device via a bypass connection. According to some embodiments, the dual layer mechanism is configured to coupled to the medical device and/or the controller such that an additional connection line is added between the controller and the medical device.

According to some embodiments, the dual layer mechanism is configured to couple to the controller and the medical device via an in-line connection, in which the dual layer architecture is positioned between one or more communication channel coupling between the controller and the medical device. According to some embodiments, the dual layer mechanism is configured to couple to the controller and the medical device via an in-line connection, in which the dual layer architecture is positioned between the only communication channel (and/or all of the communication channels) coupling between the controller and the medical device.

According to some embodiments, the medical device could also be a potential source of an attack on the system. Thus, by surrounding the system with one-way links, it is possible to guaranty the flow of information and makes the detection and prevention system itself very secure. According to some embodiments, the security of the detection and prevention system must be considered carefully, as the device and/or system may be the last line of defense to the medical device. According to some embodiments, the device and/or system is configured to be able to alert the user (such as, e.g., the operators and/or the patients) if an anomalous instruction is detected. According to some embodiments, the device is coupled to a monitor screen. According to some embodiments, the device is coupled to a monitor screen via a one-way link, thereby protecting the system from the monitor screen.

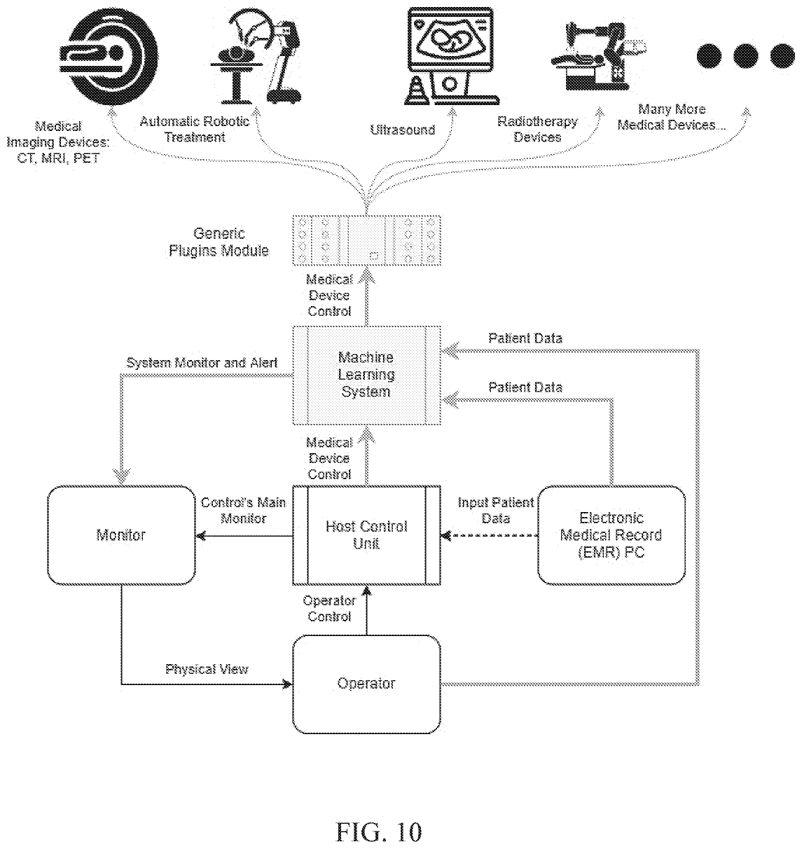

Reference is made to FIG. 10, which shows a schematic illustration of an exemplary plugin module, in accordance with some embodiments of the present invention.

According to some embodiments, the device and/or system includes a plugin module configured to couple to a medical device. According to some embodiments, the plugin module is generic. According to some embodiments, the plugin module is configured to couple to a specific medical device. According to some embodiments, the plugin module may be configured for a specific medical device. According to some embodiments, the plugin module is configured to adapt the communication between the protection device and/or system and a plurality of medical devices.

According to some embodiments, the plugin module can be tailored to the specific medical device and the specific solution that they require. According to some embodiments, the plugin module is configured to allow the protection device and/or system to adapt to a wide range of medical devices while providing the same core solution to each medical device. According to some embodiments, the plugin module is configured to allow the system to create a custom configuration for each medical device, which may include of a specified desired solution. For example, in some embodiments, the solution may include a cyber security-based solution configured to protect a CT medical device which could be used with an optimization-based solution to enhance the parameters of the instructions of the controller. According to some embodiments, the same and/or a different cyber security-based solution can be used in an MM medical device with and/or without the optimization-based solution.

According to some embodiments, the device and/or system can include multiple configurable solutions. According to some embodiments, the plugin module is configured to allow a costume solution to specific medical device based on its specific requirements.

According to some embodiments, the protective device and/or system includes a processor and a memory module. According to some embodiments, the memory module includes a software program configured to be implemented by the processor. According to some embodiments, the software program includes an algorithm for detection and prevention of anomalous instructions sent to the medical device from a controller.

### Protective Mechanism-Hypervisor

According to some embodiments, the method includes receiving the instructions using a hypervisor module. According to some embodiments, the device may include a hypervisor module.

According to some embodiments, the hypervisor module is configured to separate and/or protect the medical device from the controller by including one or more virtual machines. According to some embodiments, the hypervisor module may include the dual layer architecture. According to some embodiments, the hypervisor module may be coupled to one or more processors configured to implement the dual layer architecture on one or more instructions. According to some embodiments, the hypervisor module may be coupled to the controller of the medical device. According to some embodiments, the hypervisor module may be coupled to the controller of the medical device such that the medical device is unaware of the hypervisor being in communication with the controller.

According to some embodiments, the hypervisor module is configured to implement one or more instructions sent by the controller to the medical device, onto one or more virtual machines (VM). According to some embodiments, hypervisor module is configured to

According to some embodiments, the hypervisor module is configured to receive the instructions from the controller. According to some embodiments, the hypervisor algorithm is configured to apply the received instructions to a virtual machine instead of the medical device. For example, in some embodiments, the controller may send instructions to the medical device, however, the instructions will be implemented by the hypervisor module onto a virtual machine, thereby the instructions will not reach the medical device. According to some embodiments, the instructions are sent form the hypervisor module to the medical device only after the instructions are implemented by the one or more virtual machine, and/or applied to the dual layer architecture. According to some embodiments, the instructions are sent form the hypervisor module to the medical device only after the instructions are identified as non-anomalous (e.g., not having the instructions identified as anomalous).

According to some embodiments, there is provided a method for cyber-security risk assessment. In some embodiments, the method may be termed TLDR (Threat identification, ontology-based Likelihood, severity Decomposition, and Risk assessment). In some embodiments, the method may be used to identify and assess risks associated with cyber-attacks on medical devices, such as, CT, MM, PET, X-Ray, and the like. In some embodiments, the method is advantageous as it may provide results comparable or superior to expert assessments. According to some embodiments, the method for risk assessment may be used to identify potential medical devices cyber-security threats by decomposing the severity of cyber-attacks into several objectives (aspects), such as, for example, six objectives. In some embodiments, the risk assessment method may enable organizations to customize the risk assessments and implied priorities, using relative weights for the objectives.

According to some embodiments, the method includes assessing the attack severity by extending the severity aspects and providing additional aspects unique to specified medical devices.

According to some embodiments, the assessment method includes identifying potentially vulnerable components of MIDs using Attack Flow Diagrams (AFDs). According to some embodiments, the AFDs include diagrams of MIDs, consisting of their main components and the information flow between them. According to some embodiments, IDs of all potential attacks are included, thus identifying potential vulnerabilities. identifying potential attacks and marking them on the AFDs. According to some embodiments, the assessment method includes estimating the overall likelihood (probability) for each attack. According to some embodiments, the method includes decomposing the severity of all of the MID attacks identified into a plurality of aspects. According to some embodiments, the aspects may be categorized into two groups: device aspects and patient aspects. According to some embodiments, the device aspects may be sub-categorized into a plurality of sub-categories. According to some embodiments, the sub-categories may include availability (compromising the availability of the device (e.g., ransomware)), and integrity (compromising the integrity (or causing physical erosion) of the device (e.g., disruption of the device's motors). According to some embodiments, the patient aspects may be sub-categorized into a plurality of sub-categories. According to some embodiments, the sub-categories may include confidentiality (compromising the privacy of patients (e.g., leakage of private medical records)), clinical (affecting the clinical outcome (e.g., making an incorrect imaging diagnosis in the case of MIDs), patient harm (causing physical damage to the patient (e.g., tissue burns)), and scale (the size of the affected group (e.g., affecting a single patient or numerous patients)).

According to some embodiments, each attack has an expected specific magnitude of impact on each of the six severity aspects. According to some embodiments, the method includes assigning each severity aspect an importance weight, based, at least in part, on the organization's policies and priorities.

According to some embodiments, the method includes computing composite severity assessments for the attacks using the weighted sums of a magnitude of the impact of the six decomposed severity aspects for each attack, weighted by the organization-specific aspect weights, using Eq. (1):

Severityⱼ=Σᵢ₌₁⁶ωᵢ·sᵢⱼ+b (1)

(ωᵢ=the weight of the ith decomposed severity aspect, sᵢⱼ=assessment of the expected magnitude of the impact of the ith decomposed severity aspect for the jth specific attack, b=a potentially needed constant bias.)

According to some embodiments, the method includes computing the risk assessment for each attack, and multiplying its likelihood by its composite severity. According to some embodiments, the risk assessments can be used by organizations to prioritize the attacks and guide their efforts in mitigating the risk.

According to some embodiments, the method for cyber-security risk assessment can be used as a cyber-security risk assessment methodology while also providing additional details regarding the severity's components and supporting organizational prioritization. In addition, according to some embodiments, the TLDR methodology is easily customized to meet organizational needs, primarily by using the organization-specific relative importance weights for the severity aspects. According to some embodiments, the method enables easy, uniform assessment of new, future threats or new implications of identified attacks, which can be immediately adopted and customized by organizations. Advantageously, the method can be fine-tuned by organizations so that the predefined uniform (across all potential attacks) default weights are aligned with organizational policies and priorities. According to some embodiments, the weights can then potentially be used for attacks in other medical domains.

According to some embodiments, the method includes identifying the potentially vulnerable components of medical devices, for example, different medical imaging devices (MIDs). According to some embodiments, the method includes identifying the potential attacks. According to some embodiments, the method includes mapping the discovered attacks into a known attack ontology. According to some embodiments, the method includes estimating the likelihood of the mapped CAPECs in the medical domain with the assistance of a panel of senior healthcare Information Security Experts (ISEs). According to some embodiments, the method includes computing the CAPEC-based likelihood estimates of each attack. According to some embodiments, the method includes decomposing each attack into several severity aspects and assigning them weights. According to some embodiments, the method includes assessing the magnitude of the impact of each of the severity aspects for each attack with the assistance of a panel of senior Medical Experts (MEs). According to some embodiments, the method includes computing the composite severity assessments for each attack. According to some embodiments, the method includes integrating the likelihood and severity of each attack into its risk, and thus prioritizing it.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods according to some embodiments may be described in a specific sequence, methods of the disclosure may include some or all of the described stages carried out in a different order. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1—Data Collection

In total, 8,277 instructions were collected from 2,643 different Studies (which is roughly the number of patients) and were then separated into a train set of 6,286 (75%) instructions and a test set of 1,991 (25%) instructions.

The test set includes 1,312 normal instructions and 679 anomalous instructions. Collecting labeled anomalous instructions (e.g., malicious instructions due to a cyber-attack) is very difficult since anomalous instructions are rare and unlabeled (i.e., the metadata does not include an anomaly label or whether the instruction satisfied the clinical objective). Following, 679 CF and CS anomalous instructions were collected/detected.

### CF Anomalous Instructions Collection/Detection

While analyzing the collected instructions, 216 instructions appeared suspicious/abnormal (which were labeled as a Physics Procedure for the Study meta-data). A technical discussion with the manufacturer verified that these instructions were part of a technical maintenance calibration procedure and should not be used on patients; thus, these instructions were considered as CF anomalous instructions. In addition, 59 malicious anomalous instructions were manually recorded 59 by asking an expert operator to, intentionally, execute malicious instructions (e.g., high radiation, high motor speed, long scan time, etc.) on a CT scanner (without a patient). These anomalous instructions are CF, as they should not be sent regardless of the patient being scanned or the clinical objective. In total, 275 CF anomalous instructions were collected. CS anomalous instructions collection/detection:

While analyzing the collected instructions, 140 Studies (containing a total of 404 instructions), which make up 5% of all non-anomalous instructions, were repeated twice, one after the other, for the same patient, for no apparent reason; while there could be many reasons for repeating a Study, a repetition may indicate that the first Study did not satisfy the clinical objective. Unlike the CF anomalous instructions, the repeated instructions are, in fact, normal instructions that are only considered anomalous given the clinical objective context; thus, these 5% (i.e., 404 instructions) repeated instructions were considered as CS anomalous instructions. In total, 404 CS anomalous instructions were collected.

Data preprocessing: For each algorithm training, the data was cleaned (e.g., removed instructions that include parameters with NaN value (not a number)), encoded categorical features (one-hot encoding was used for neural networks), and applied standardization (i.e., Z-score normalization). Also, basic feature selection algorithms were used to drop features with a single value and features with a 100% correlation with other features. For the supervised CS layer, instructions of labels with less than 100 examples were dropped.

### Example 2—Evaluation of Performances of the First Detection Layer and the Second Detection Layer

Implementation: For each layer, the algorithm with the highest F1 score on the test set without the CS anomalous instructions was selected.

Evaluation: The performance, with respect to overall anomalous instructions detection (both CF and CS), of (1) just the CF layer (representing the capabilities of current state-of-the-art unsupervised anomaly detection) was compared to (2) the performance of the overall anomalous instructions detection when using, in addition to the first layer, also the CS layer.

The context-free (CF) layer. The first layer receives the instructions (without context) as input and uses a pre-trained (using non-anomalous instructions) unsupervised anomaly detection algorithm to detect CF anomalous instructions and alert the operator. However, instructions that were not detected as anomalous might still be CS anomalous within a particular context; in order to detect these, the second layer is used. Implementation: 11 state-of-the-art unsupervised anomaly detection algorithms (listed in Table 1) were used, some of them were implemented by the PyOD python toolbox. Each algorithm calculates the anomaly score of an instruction, and if it is above the anomaly threshold, the instruction is detected as anomalous. The anomaly threshold is the 100. (1−contamination) percentile of the training set anomaly scores, where contamination represents the expected pro-portion of anomalies in the data and is given during the initialization of the algorithms. Three ensembles composed of the top (in terms of highest F1 score in the initial evaluation) four algorithms from the 11 algorithms that were evaluated were added (i.e., Angle-based Outlier Detector(ABOD),k-NN, One-Class Support Vector Machine (OCSVM), and Isolation Forest (IForest)): the Locally Selective Combination of Parallel Outlier Ensembles (LSCP), and two that chooses either the maximal or the average anomaly score of these four algorithms as the final anomaly score of the ensemble (which is compared to the ensemble's anomaly threshold, to determine its output, as is the case in the other algorithms).

Evaluation:

The new architecture in the computed tomography (CT) domain was evaluated, using 8,277 CT instructions that were recorded. The CF layer was evaluated using 14 different unsupervised anomaly detection algorithms. The CS layer, for four different types of clinical objective contexts, was evaluated using five supervised classification algorithms for each context. Adding the second CS layer to the architecture improved the overall anomaly detection performance from an F1 score of 71.6% (using only the CF layer) to 82.3%-98.8% (depending on the clinical objective used). Furthermore, the CS layer enables the detection of CS anomalies, using the semantics of the device's procedure, which cannot be detected using only the purely syntactic CF layer.

Since 275 CF anomalous instructions were collected out of a total of 8,277 collected instructions, a contamination parameter of 0.01 (slightly lower than the actual portion of anomalies in the data) seemed to work well for most algorithms; thus, the decision was made to use it throughout the evaluation. The algorithms were trained using non-anomalous instructions and the performance was evaluated using the CF and CS anomalous instructions. The evaluation of just the CF anomalous instructions was included in order to show the performance of just on this type of anomalous instructions. The performance was evaluated using the confusion matrix, accuracy, recall, precision, and F1 score.

Implementation five state-of-the-art multi-class classification algorithms (listed in FIG. 5) were used for the detection of CS anomalous instructions and implemented by the scikit-learn classification library.

Evaluation. Each of the four scan type hierarchical abstraction levels of the clinical objective

contexts were evaluated separately, using the five supervised classification algorithms. The algorithms were trained using non-anomalous labeled instructions and the performance was evaluated using the test set without the anomalous instructions. The comparison between the predictions of a CS instruction and the intended contexts (given as its input) should be True for non-anomalous instructions, and False for CS instructions (note that that the first layer already discarded CF instructions). Therefore, the performance was evaluated using the diagonal of the multi-class confusion matrix (representing the correctly classified instructions), accuracy, and weighted F1 score (due to class imbalance). The evaluation of just the CS anomalous instructions (which are not part of the train or test sets) was included to show the performance of CS anomalous instructions detection. The comparison between the predictions of the correctly classified CS instructions will result in a set of contexts that will (correctly) not match the intended contexts.

The dual-layer protection algorithm was applied to a CT scanner device in order to test it. The clinical objective context was a main focus of this specific study.

The CF layer. In the exemplary results depicted by FIG. 5, the performance on the CF anomalous instructions are high for several algorithms, and highest for the Ensemble Average algorithm. However, the performance on the CF and CS anomalous instructions are much lower, since the algorithms fail to detect the CS anomalous instructions. Note that the number of instructions that were used is lower than the number of collected instructions due to the preprocessing; for example, Surview type instructions were removed, as these types of instructions are quick initial scans that have a lower potential of damage.

FIG. 5 shows (exemplary) results of the unsupervised anomaly detection for the CF layer. The training set included 3,595 non-anomalous instructions, and the test set included 481 anomalous instructions (275 of which are CF) and 764 non-anomalous instructions.

The CS layer. For this layer, the given instructions can be assumed as not CF anomalous because the CF layer already detected them. In this study, the clinical objective CS anomalous instructions were evaluated (patient context is beyond the scope of the current specific study). The clinical objective is represented using the four hierarchical abstractions of the scan type and is used as the target labels of the supervised classification algorithms that are trained. For each clinical context, the performance on the test set (i.e., not including anomalous instructions) and on the test set with the CS anomalous instructions is evaluated. Note that the preprocessing is slightly different since only instructions of labels with at least 100 examples were used.

Scan Options Objective. In the exemplary results depicted in FIG. 6A, the supervised classification appears to work extremely well for this level of abstraction, with most algorithms reaching an F1 score of 1. This implies that the topmost level of the scan type hierarchical separates instructions very well. Note that for this classification, the Surview instructions were not removed during the preprocessing.

Body Part Objective. In the exemplary results depicted in FIG. 6B, the supervised classification appears to work well for this level of abstraction, too, with some algorithms reaching an F1 score of 1. Note that the number of instructions is lower since not all instructions include the Body Part label, and instructions with Surview Scan Option were removed.

Study Objective. In the exemplary results depicted in FIG. 6C, the performance of the supervised classification algorithms decreased in comparison to higher-level abstractions, with a maximal F1 score of 0.895 for Random Forest (RF). Note that classes with a relative high number of instructions available during training have a higher F1 score, implying that more data might improve the performance. Furthermore, from the evaluation of the confusion matrix it appears that the wrong classification was mostly between relatively similar Study types; for example, the Random Forest classifier was confused between Abdomen, Chest, and Chest/Abdomen, however, was not confused between Abdomen and Head or Abdomen and CTA Cardiac. Protocol Objective. In the exemplary results depicted in FIG. 6D, the performance of the supervised classification algorithms decreased in comparison to higher-level abstractions, with a maximal F1 score of 0.819 for RF. Similar to the Study class, classes with a relative high number of instruction available during training have a higher F1 score, and the wrong classifications were mostly between relatively similar Protocols. In the exemplary results depicted in FIG. 7, it is shown that adding the second CS layer improved the overall performance (F1 score and accuracy) for each clinical objective, relative to the performance of the CS layer. Notably, while the detection of CS anomalous instructions was improved, miss-classification of the non-anomalous instructions (which are also analyzed by this layer) resulted in increased false positives. Additionally, in one example, using only the CF layer led to detecting only 57.2% of the anomalous instructions, while adding the CS layer and knowledge of the Study clinical objective led to a sensitivity of 94.7%.

FIG. 7 shows the exemplary results of a dual-layer architecture, showing the performance of the CF layer alone, and with the additional second CS layers. For the first CF layer, the Ensemble Average algorithms were used. For each second CS layer, the RF classifier was used (respectively).

The dual-layer architecture for the protection of medical devices from CF and CS anomalous instructions was evaluated for its performance using CT host PC instructions (that were collected from an operational CT at a hospital), for four, hierarchical, scan type abstractions of the clinical objective context.

The CF layer detected all 275/275 CF anomalous instructions using unsupervised anomaly detection methods (e.g., ensemble average algorithm); however, it failed to detect the 206 CS anomalous instructions, resulting in an F1 score of 0.716. The CS layer detected 82%-100% of the CS anomalous instructions (depending on the clinical context used) by comparing supervised classification methods' predictions (e.g., RF) to the real context (received from a separated secure private channel); However, the low performance of some classifiers increased the false positive rate (FPR) due to wrong classifications of non-anomalous instructions. Accordingly, it may be concluded that adding the second CS layer increased the overall F1 score from 71.6% to82.3%-98.8%.

From the results, it can be concluded that for higher-level abstractions (i.e., Scan Options and Body Part) the CS layer performed very well with an F1 score of 99.4%-100%, while for lower-level abstractions (i.e. Study and Protocol) the performance was lower with an F1 score of 81.9%-89.5%. One reason for this is that lower-levels in the hierarchy limited the amount of available training data for each class; for example, for Study context RF classifier, the F1 score for CTA Cardiac class (trained using 854 instructions) was 0.96, compared with 0.842 for CTA Head class (trained using 269 instructions). Furthermore, from the evaluation of the confusion matrices of classifiers of lower-levels in the hierarchy, it was discovered that wrong classification was given mostly to relatively similar classes (e.g., between Abdomen Routine (C+)/Abdomen and Abdomen Routine (C−)/Abdomen classes of Protocol). While such classifications are considered wrong, there might not be a real significant difference between such classes. Therefore, by merging such classes, the amount of available training data for the merged class is increased and the number of wrong classifications between similar classes is reduced.

FIG. 6A, 6B, 6C, and 6D show the exemplary results of the supervised classification of clinical objective contexts for the CS layer on the test set and the CS anomalous instructions, including the per-class F1 score, the total accuracy, and the total weighted F1 average. At the bottom of each table, the number of instructions used during training and testing is presented, per-class.

In conclusion, it has been demonstrated herein that, for medical devices, such as, for example, CT devices, the dual-layer architecture for protection of medical devices is effective and reliable.

1.-56. (canceled)

57. A method for detection of anomalous instructions sent from a controller to be received by a medical device, the method comprising:
receiving instructions sent from the controller, said instruction being intended to be received by the medical device; and
analyzing the instructions by applying:
a first detection layer, said first detection layer comprising an unsupervised machine learning model configured to detect context free (CF) anomalous instructions; and
a second detection layer, said second detection layer comprising a supervised machine learning model configured to detect context sensitive (CS) anomalous instructions;
wherein the second layer is applied to instructions that were not detected as anomalous by the first detection layer.

58. The method according to claim 57, wherein the first detection layer and the second detection layer are applied in series, wherein the detection of said anomalous instructions is performed in real time.

59. The method according to claim 57, wherein analyzing by applying the first detection layer comprises calculating an anomaly score of the received instructions and comparing the anomaly score with an anomaly threshold.

60. The method according to claim 59, wherein the comparison between the anomaly score and the anomaly threshold is associated with one or more of: a deviation from a predetermined threshold value, a deviation from a corresponding standard parameter value, an unlikely parameter value, and an unlikely combination of parameter value.

61. The method according to claim 57, wherein applying the first layer comprises determining if one or more parameter values of the received instructions deviate from values of corresponding parameters of a predetermined parameter value data set, wherein a deviation between the one or more parameter values of the received instructions and values of parameters in the predetermined value data set is indicative of the instructions being anomalous.

62. The method according to claim 57, wherein the context sensitive (CS) anomalous instructions relate to one or more context values associated with the received instructions and to a specific patient intended to be monitored or treated by the medical device by implementing the received instructions.

63. The method according to claim 57, further comprising receiving a context value associated with the received instructions, and wherein analyzing by applying the second detection layer comprises:
applying the received instructions to at least one supervised classification algorithm configured to output a predicted context value associated with the received instructions; and
comparing the predicted context value with the received context value.

64. The method according to claim 57, wherein the medical device is a medical imaging device (MID), selected from CT, MRI, X-Ray generator (digital radiography), Ultrasound, SPECT, and PET; and wherein the controller comprises a host PC of a medical device.

65. The method according to claim 57, further comprising issuing an alert if anomalous instruction(s) have been identified and/or preventing or blocking a detected anomalous instruction from reaching the medical device.

66. The method according to claim 57, comprising generating an anomaly explanation output, wherein the output is configured to provide a user an explanation associated with a reason for instructions being detected as anomalous.

67. The method according to claim 57, comprising generating one or more revisions to the anomalous instructions utilizing a revision suggestion module and/or further comprising assigning a risk score to the detected anomalous instructions associated with a severity level of the detected anomalous instructions.

68. The method according to claim 57, wherein said instructions are received using a hypervisor module configured to apply the received instructions to at least one virtual machine (VM).

69. A device for detection of anomalous instructions sent form a controller to a medical device, the device comprising:
a processor configured to:
receive instructions from the controller, said instructions being intended to be received by the medical device; and
analyze the instructions by applying:
a first detection layer comprising an unsupervised detection layer machine learning model configured to detect context free (CF) anomalous instructions; and
a second detection layer comprising a supervised detection layer machine learning model configured to detect context sensitive (CS) anomalous instructions;
wherein the first and second detection layers are applied consecutively, whereby the second layer is applied to instructions that were not detected as anomalous by the first detection layer.

70. The device according to claim 69, wherein the first detection layer and the second detection layer are applied in series, wherein the detection of said anomalous instructions is performed in real time.

71. The device according to claim 69, wherein the processor is further configured to issue an alert if anomalous instruction(s) have been identified and/or preventing or blocking a detected anomalous instruction from reaching the medical device.

72. The device according to claim 69, comprising an anomaly explanation module configured to generate an output, wherein the output provides an operator/user an explanation associated with a reason for instructions being detected as anomalous; and/or a revision suggestion module configured to generate one or more revisions to the anomalous instructions.

73. The device according to claim 69, further comprising one or more of: a communication unit, a power source, a display, a user interface, an alert unit.

74. The device according to claim 69, wherein the device is configured to couple to the controller at a first end thereof and couple to the medical device at a second end thereof; and/or wherein the device is further configured to wirelessly communicate with at least one of the controllers and the medical device.

75. The device according to claim 69, comprising a hypervisor module configured to receive the instructions and apply the received instructions to a virtual machine (VM); and/or at least one unidirectional channel coupled to the processor and configured to direct the instructions in only one direction, thereby preventing one or more signals from traveling from the processor to an external device.

76. A non-transitory computer-readable medium having stored thereon instructions that cause a processor to:
receive instructions sent from a controller, said instructions being intended to be received by a medical device; and
analyze the instructions by applying:
a first detection layer to the received instructions, said first detection layer being an unsupervised detection layer comprises machine learning model configured to detect context free (CF) anomalous instructions; and
a second detection layer, said second detection layer being a supervised detection layer comprises machine learning model, configured to detect context sensitive (CS) anomalous instructions;
wherein the second layer is applied to instructions that were not detected as anomalous by the first detection layer.
